(19) United States
(12) Patent Application Publication
(10) Pub. No.: US 2024/0416106 A1
(43) Pub. Date: Dec. 19, 2024

(54) ENERGY GENERATION IN A BLOOD PUMP FROM AN ALTERNATING OR ROTATING MAGNETIC FIELD

(71) Applicant: Maquet Cardiopulmonary GmbH, Rastatt (DE)

(72) Inventors: Andreas BANK, Rastatt (DE); Uwe LUTZ, Remchingen (DE); Benjamin KOMM, Ottersweier (DE)

(21) Appl. No.: 18/689,664

(22) PCT Filed: Sep. 7, 2022
(86) PCT No.: PCT/EP2022/074855

§ 371 Date: Mar. 6, 2024

Related U.S. Application Data
(60) Provisional application No. 63/241,326, filed on Sep. 7, 2021.

(51) Int. Cl. A61M 60/422; A61M 1/36; A61M 60/113; A61M 60/226; A61M 60/38; A61M 60/538; A61M 60/546; A61M 60/554; F04D 13/06; H02K 35/02

(52) U.S. Cl. CPC A61M 60/422; A61M 1/3666; A61M 60/113; A61M 60/226; A61M 60/38; A61M 60/538; A61M 60/546; A61M 60/554; F04D 13/06; H02K 35/02

(57) ABSTRACT

An extracorporeal blood flow system includes a blood pump comprising a pump rotor coupled to be rotated by a motor, a transducer disposed in operative proximity to the rotor and configured to generate electrical energy in response to a first changing magnetic field associated with rotation of the rotor, and at least one sensor powered by electrical energy generated by the transducer.

### CROSS-REFERENCE TO RELATED APPLICATION

The present application claims priority of U.S. Provisional Patent Application No. 63/241,326, filed on Sep. 7, 2021, the disclosure of which is hereby incorporated by reference in its entirety.

### BACKGROUND OF THE DISCLOSURE

### Field of the Disclosure

The present disclosure relates to harvesting electrical energy from an alternating or rotating magnetic field generated by a motor. In some embodiments, the present disclosure relates to a system and method for harvesting electrical energy from an alternating or rotating magnetic field generated by an electric blood pump of an extracorporeal blood circulation system and powering electronic devices with the harvested electrical energy.

### Description of Related Art

Cardiopulmonary bypass is a technique in which an extracorporeal device is used to circumvent a patient's native heart and lungs during a medical procedure, such as during open heart surgery and as a treatment for acute respiratory distress syndrome (ARDS). Examples of extracorporeal devices include cardiopulmonary bypass machines and extracorporeal membrane oxygenation (ECMO) machines. To mimic the function of the patient's native lungs and heart, such extracorporeal devices include an oxygenator that exposes extracted blood to oxygen and removes carbon dioxide, and a blood pump that circulates the blood through the oxygenator and back to the patient's circulatory system. Various sensors associated with the oxygenator may be used to gather data and to provide information used to control operating parameters of the extracorporeal device. In conventional systems, such sensors are typically powered by a power source that is remotely mounted from the oxygenator. As such, a power cable is connected between the power source and these sensors by a technologist during setup of the extracorporeal device, which adds complexity and bulkiness to such systems. In addition to increasing setup time of the extracorporeal device, connection of the power cable may be inadvertently forgotten or performed improperly, adversely affecting performance of the medical procedure. In view of the above, there exists a need for alternative means for powering various sensors of an extracorporeal blood circulation system and/or other electronic components of extracorporeal devices in order to simplify set-up and operation of extracorporeal blood circulation systems. Of course, the technology of the present disclosure may be applied more broadly to fluid circulation systems in general and is not limited to blood circulation systems. In fact, the technology of the present disclosure is applicable to any system having a pump or other device that generates a changing magnetic field from which electrical energy may be harvested and used to power one or more electronic devices of the system.

In the electrical arts unrelated to extracorporeal devices, Wiegand sensors, first developed in the 1970s, have been used to measure position and speed, particularly rotational position and speed, of electromechanical components. Wiegand sensors operate by generating a voltage pulse in response to a changing magnetic field. The voltage pulses generated by the Wiegand sensor can be counted, typically by an electronic processor, to determine the number and timing of changes to the magnetic field. This data can in turn be correlated to a position and/or a rotational speed of the electromechanical component. As of the present time, Wiegand sensors have not been used as a means of electrical power generation by harvesting electrical energy from changing magnetic fields associated with an electric motor in order to supply electrical power to other electrical components in the field of fluid flow systems.

### SUMMARY OF THE DISCLOSURE

In view of the foregoing, the present disclosure is directed to devices, systems, and methods for providing electrical power to sensors and/or other electrical components by harvesting energy from an alternating/rotating magnetic field generated by a pump or other device comprising an electric motor.

Non-limiting, illustrative Embodiment 1 of the present disclosure relates to an extracorporeal blood flow system including a blood pump including a pump rotor coupled to be rotated by an electric motor, a transducer disposed in operative proximity to the pump rotor and configured to generate electrical energy in response to a first changing magnetic field associated with rotation of the pump rotor, and at least one sensor. The at least one sensor is powered by electrical energy generated by the transducer.

Non-limiting, illustrative Embodiment 2 of the present disclosure further modifies non-limiting Embodiment 1, and provides that the motor includes a motor rotor and motor stator. The motor rotor is configured to rotate relative to the motor stator in response to a second changing magnetic field associated with the motor stator and the motor rotor.

Non-limiting, illustrative Embodiment 3 of the present disclosure further modifies non-limiting Embodiments 1 or 2, and provides that the transducer is disposed in operative proximity to the motor stator and the motor rotor to generate electrical energy in response to the second changing magnetic field associated with the motor stator and the motor rotor.

Non-limiting, illustrative Embodiment 4 of the present disclosure further modifies any of non-limiting Embodiments 1 to 3, and provides that at least one of the motor rotor and the motor stator includes permanent magnets, at least one of the motor rotor and the motor stator includes electromagnets, and the motor is configured to periodically change the polarity of the electromagnets to cause rotation of the motor rotor.

Non-limiting, illustrative Embodiment 5 of the present disclosure further modifies any of non-limiting Embodiments 1 to 4, and provides that the transducer includes a Wiegand inductor.

Non-limiting, illustrative Embodiment 6 of the present disclosure further modifies any of non-limiting Embodiments 1 to 5, and provides that the transducer includes a dynamo.

Non-limiting, illustrative Embodiment 7 of the present disclosure further modifies any of non-limiting Embodiments 1 to 6, and further includes an oxygenator including the at least one sensor.

Non-limiting, illustrative Embodiment 8 of the present disclosure further modifies any of non-limiting Embodiments 1 to 7, and provides that the at least one sensor includes at least one of a blood temperature sensor, a blood pressure sensor, a flow rate sensor, and a distance sensor.

Non-limiting, illustrative Embodiment 9 of the present disclosure further modifies any of non-limiting Embodiments 1 to 8, and provides that the rotor is magnetically coupled to the electric motor of the blood pump so as to generate the changing magnetic field when the rotor is rotated.

Non-limiting, illustrative Embodiment 10 of the present disclosure further modifies any of non-limiting Embodiments 1 to 9, and provides that a drive shaft couples the rotor to the electric motor of the blood pump so as to rotate the rotor to generate the changing magnetic field when the rotor is rotated.

Non-limiting, illustrative Embodiment 11 of the present disclosure further modifies any of non-limiting Embodiments 1 to 10, and further includes a controller programmed or configured to receive an output data signal from the at least one sensor.

Non-limiting, illustrative Embodiment 12 of the present disclosure further modifies any of non-limiting Embodiments 1 to 11, and provides that the at least one sensor is configured to wirelessly transmit the output data signal to the controller.

Non-limiting, illustrative Embodiment 13 of the present disclosure further modifies any of non-limiting Embodiments 1 to 12, and further includes a heart-lung machine including the controller.

Non-limiting, illustrative Embodiment 14 of the present disclosure further modifies any of non-limiting Embodiments 1 to 13, and provides that the electrical energy generated by the transducer includes a plurality of first voltage pulses. Each first voltage pulse has a substantially constant voltage unaffected by changes to a rotational speed of the first changing magnetic field.

Non-limiting, illustrative Embodiment 15 of the present disclosure further modifies any of non-limiting Embodiments 1 to 14, and provides that the electrical energy generated by the transducer includes a plurality of first voltage pulses. Each first voltage pulse has a substantially constant voltage unaffected by changes to a rotational speed of the first and second changing magnetic fields.

Non-limiting, illustrative Embodiment 16 of the present disclosure further modifies any of non-limiting Embodiments 1 to 15, and provides that the electrical energy generated by the transducer includes a plurality of second voltage pulses. Each second voltage pulse having a substantially constant voltage unaffected by changes to the rotational speed of the first and second changing magnetic fields. The first voltage pulses are substantially different from the second voltage pulses.

Non-limiting, illustrative Embodiment 17 of the present disclosure further modifies any of non-limiting Embodiments 1 to 16, and further includes a conversion box for storing and conditioning the electrical energy generated by the transducer so that the electrical energy generated by the transducer is in a form suitable to power the at least one sensor.

Non-limiting, illustrative Embodiment 18 of the present disclosure further modifies any of non-limiting Embodiments 1 to 17, and provides that the system is a medical device selected from the group consisting of a cardiopulmonary bypass machine, an extracorporeal membrane oxygenation machine, and a pump assisted lung protection machine.

Non-limiting, illustrative Embodiment 19 of the present disclosure is directed to a fluid flow system including a fluid pump including a pump rotor coupled to be rotated by an electric motor, a transducer disposed in operative proximity to the rotor and configured to generate electrical energy in response to a first changing magnetic field associated with rotation of the pump rotor, and at least one sensor. The at least one sensor is powered by electrical energy generated by the transducer.

Non-limiting, illustrative Embodiment 20 of the present disclosure further modifies non-limiting Embodiment 19, and provides that the motor includes a motor rotor and motor stator, the motor rotor configured to rotate relative to the motor stator in response to a second changing magnetic field associated with the motor stator and the motor rotor.

Non-limiting, illustrative Embodiment 21 of the present disclosure further modifies non-limiting Embodiments 19 or 20, and provides that the transducer is disposed in operative proximity to the motor stator and the motor rotor to generate electrical energy in response to the second changing magnetic field associated with the motor stator and the motor rotor.

Non-limiting, illustrative Embodiment 22 of the present disclosure further modifies any of non-limiting Embodiments 19 to 21, and provides that at least one of the motor rotor and the motor stator includes permanent magnets, at least one of the motor rotor and the motor stator includes electromagnets, and the motor is configured to periodically change the polarity of the electromagnets to cause rotation of the motor rotor.

Non-limiting, illustrative Embodiment 23 of the present disclosure further modifies any of non-limiting Embodiments 19 to 22, and provides that the transducer includes a Wiegand inductor.

Embodiment 24 of the present disclosure further modifies any of non-limiting Embodiments 19 to 23, and provides that the transducer includes a dynamo.

Non-limiting, illustrative Embodiment 25 of the present disclosure further modifies any of non-limiting Embodiments 19 to 24, and provides that the at least one sensor includes at least one of a fluid temperature sensor, a fluid pressure sensor, a flow rate sensor, and a distance sensor.

Non-limiting, illustrative Embodiment 26 of the present disclosure further modifies any of non-limiting Embodiments 19 to 25, and provides that the rotor is magnetically coupled to the electric motor of the fluid pump so as to generate the changing magnetic field when the rotor is rotated.

Non-limiting, illustrative Embodiment 27 of the present disclosure further modifies any of non-limiting Embodiments 19 to 26, and provides that a drive shaft couples the rotor to the electric motor of the blood pump so as to rotate the rotor to generate the changing magnetic field when the rotor is rotated.

Non-limiting, illustrative Embodiment 28 of the present disclosure further modifies any of non-limiting Embodiments 19 to 27, and further includes a controller programmed or configured to receive an output data signal from the at least one sensor.

Non-limiting, illustrative Embodiment 29 of the present disclosure further modifies any of non-limiting Embodiments 19 to 28, and provides that the at least one sensor is configured to wirelessly transmit the output data signal to the controller.

Non-limiting, illustrative Embodiment 30 of the present disclosure further modifies any of non-limiting Embodiments 19 to 29, and provides that the electrical energy generated by the transducer includes a plurality of first voltage pulses. Each first voltage pulse has a substantially constant voltage unaffected by changes to a rotational speed of the first changing magnetic field.

Non-limiting, illustrative Embodiment 31 of the present disclosure further modifies any of non-limiting Embodiments 19 to 30, and provides that the electrical energy generated by the transducer includes a plurality of first voltage pulses. Each first voltage pulse has a substantially constant voltage unaffected by changes to a rotational speed of the first and second changing magnetic fields.

Non-limiting, illustrative Embodiment 32 of the present disclosure further modifies any of non-limiting Embodiments 19 to 31, and provides that the electrical energy generated by the transducer includes a plurality of second voltage pulses. Each second voltage pulse has a substantially constant voltage unaffected by changes to the rotational speed of the first changing magnetic field. The first voltage pulses are substantially different from the second voltage pulses.

Non-limiting, illustrative Embodiment 33 of the present disclosure further modifies any of non-limiting Embodiments 19 to 32, and further includes a conversion box for storing and conditioning the electrical energy generated by the transducer so that the electrical energy generated by the transducer is in a form suitable to power the at least one sensor.

Non-limiting, illustrative Embodiment 34 of the present disclosure further modifies any of non-limiting Embodiments 19 to 33, and provides that the fluid pump is a blood pump and the system is a medical device selected from the group consisting of a cardiopulmonary bypass machine, an extracorporeal membrane oxygenation machine, a pump assisted lung protection machine, and a hemodialysis machine.

Non-limiting, illustrative Embodiment 35 of the present disclosure is directed to a method of generating electrical energy to power at least one electronic device. The method includes the steps of: in a fluid pump including a magnetic field coupling a pump rotor and an electric motor, rotating the pump rotor to generate a first changing magnetic field associated with the pump rotor; inducing a voltage in a transducer as a result of rotating the first magnetic field, wherein the transducer includes a Wiegand inductor and the induced voltage includes a plurality of first voltage pulses, each first voltage pulse having a substantially constant voltage unaffected by changes to a rotational speed of the first changing magnetic field when rotating the first changing magnetic field; and powering the at least one electronic device using the induced voltage as a source of electrical energy.

Non-limiting, illustrative Embodiment 36 of the present disclosure further modifies non-limiting Embodiment 35, and provides that the electric motor includes a motor rotor and motor stator. The motor rotor is configured to rotate relative to the motor stator in response to a second changing magnetic field associated with the motor stator and the motor rotor. The method further includes the step of generating the second changing magnetic field so as to rotate the motor rotor relative to the motor stator.

Non-limiting, illustrative Embodiment 37 of the present disclosure further modifies non-limiting Embodiments 35 or 36, and further includes inducing an additional voltage in the transducer as a result of rotating the second changing magnetic field.

Non-limiting, illustrative Embodiment 38 of the present disclosure further modifies any of non-limiting Embodiments 35 to 37, and provides that at least one of the motor rotor and the motor stator includes permanent magnets, at least one of the motor rotor and the motor stator includes electromagnets, and the electric motor is configured to periodically change the polarity of the electromagnets to cause rotation of the motor rotor.

Non-limiting, illustrative Embodiment 39 of the present disclosure further modifies any of non-limiting Embodiments 35 to 38, and provides that the induced voltage includes a plurality of second voltage pulses. Each second voltage pulse has a substantially constant voltage unaffected by changes to the rotational speed of the first changing magnetic field. The first voltage pulses are substantially different from the second voltage pulses.

Non-limiting, illustrative Embodiment 40 of the present disclosure further modifies any of non-limiting Embodiments 35 to 39, and further includes the step of conditioning the electrical energy generated by the transducer. The electrical energy is conditioned with a conversion box so that the induced voltage generated by the transducer is in a form suitable to power the at least one electronic device.

Non-limiting, illustrative Embodiment 41 of the present disclosure further modifies any of non-limiting Embodiments 35 to 40, and provides that the fluid pump is a blood pump and the system is a medical device selected from the group consisting of a cardiopulmonary bypass machine, an extracorporeal membrane oxygenation machine, a pump assisted lung protection machine, and a hemodialysis machine. The at least one electronic device powered by the electrical energy is selected from the group consisting of a blood temperature sensor, a blood flow sensor, a blood pressure sensor, and a distance sensor.

Non-limiting, illustrative Embodiment 42 of the present disclosure is directed to an electric system including an electric motor including a rotor coupled to be rotated by the electric motor, a transducer disposed in operative proximity to the rotor and configured to generate electrical energy in response to a first changing magnetic field associated with rotation of the rotor, and at least one electronic device. The at least one electronic device is powered by electrical energy generated by the transducer.

Non-limiting, illustrative Embodiment 43 of the present disclosure further modifies non-limiting Embodiment 42, and provides that the electric motor includes a motor rotor and motor stator. The motor rotor is configured to rotate relative to the motor stator in response to a second changing magnetic field associated with the motor stator and the motor rotor.

Non-limiting, illustrative Embodiment 44 of the present disclosure further modifies non-limiting Embodiments 42 or 43, and provides that the transducer is disposed in operative proximity to the motor stator and the motor rotor to generate electrical energy in response to the second changing magnetic field associated with the motor stator and the motor rotor.

Non-limiting, illustrative Embodiment 45 of the present disclosure further modifies any of non-limiting Embodiments 42 to 44, and provides that at least one of the motor rotor and the motor stator includes permanent magnets, at least one of the motor rotor and the motor stator includes electromagnets, and the motor is configured to periodically change the polarity of the electromagnets to cause rotation of the motor rotor.

Non-limiting, illustrative Embodiment 46 of the present disclosure further modifies any of non-limiting Embodiments 42 to 45, and provides that the transducer includes a Wiegand inductor.

Non-limiting, illustrative Embodiment 47 of the present disclosure further modifies any of non-limiting Embodiments 42 to 46, and provides that the electrical energy generated by the transducer includes a plurality of first voltage pulses. Each first voltage pulse has a substantially constant voltage unaffected by changes to a rotational speed of the first changing magnetic field.

Non-limiting, illustrative Embodiment 48 of the present disclosure further modifies any of non-limiting Embodiments 42 to 47, and provides that the electrical energy generated by the transducer includes a plurality of first voltage pulses. Each first voltage pulse has a substantially constant voltage unaffected by changes to a rotational speed of the first and second changing magnetic fields.

Non-limiting, illustrative Embodiment 49 of the present disclosure further modifies any of non-limiting Embodiments 42 to 48, and provides that the electrical energy generated by the transducer includes a plurality of second voltage pulses. Each second voltage pulse has a substantially constant voltage unaffected by changes to the rotational speed of the first changing magnetic field. The first voltage pulses are substantially different from the second voltage pulses.

Non-limiting, illustrative Embodiment 50 of the present disclosure further modifies any of non-limiting Embodiments 42 to 49, and further includes a conversion box for storing and conditioning the electrical energy generated by the transducer so that the electrical energy generated by the transducer is in a form suitable to power the at least one electronic device.

Non-limiting, illustrative Embodiment 51 of the present disclosure further modifies any of non-limiting Embodiments 42 to 50, and provides that the electric motor is a component of a blood pump and the system is a medical device selected from the group consisting of a cardiopulmonary bypass machine, an extracorporeal membrane oxygenation machine, a pump assisted lung protection machine, and a hemodialysis machine. The at least one electronic device powered by the electrical energy is selected from the group consisting of a blood temperature sensor, a blood flow sensor, a blood pressure sensor, and a distance sensor.

Further details and advantages of the various non-limiting examples described in detail herein will become clear upon reviewing the following detailed description of the various non-limiting examples in conjunction with the accompanying drawing figures.

### BRIEF DESCRIPTION OF THE DRAWINGS

FIG. 1A is a perspective view of an extracorporeal circulation system in accordance with an embodiment of the present disclosure;

FIG. 1B is a schematic diagram of an extracorporeal circulation system in accordance with an embodiment of the present disclosure;

FIG. 2A is a partial schematic view of the extracorporeal circulation system of FIG. 1B in accordance with an embodiment of the present disclosure;

FIG. 2B is a partial schematic view of the extracorporeal circulation system of FIG. 1B in accordance with an embodiment of the present disclosure;

FIG. 3A is a top view of a blood pump including an associated transducer in accordance with an embodiment of the present disclosure;

FIG. 3B is a schematic top view of the blood pump of FIG. 3A, illustrating a rotating magnetic field produced during operation of the blood pump;

FIG. 3C is a side cross-sectional view of the blood pump of FIG. 3A according to an embodiment of the present disclosure;

FIG. 3D is a transverse cross-sectional view of the blood pump of FIG. 3C taken along section line II-II of FIG. 3C;

FIG. 3E is a side cross-sectional view of the blood pump of FIG. 3A according to an embodiment of the present disclosure;

FIG. 3F is a transverse cross-sectional view of the blood pump of FIG. 3C taken along section line III-III of FIG. 3C;

FIG. 4A is a partial schematic diagram of an extracorporeal circulation system in accordance with an embodiment of the present disclosure;

FIG. 4B is a partial schematic diagram of an extracorporeal circulation system in accordance with an embodiment of the present disclosure;

FIG. 5 is a partial schematic diagram of an extracorporeal circulation system in accordance with an embodiment of the present disclosure;

FIGS. 6A-6F are schematic illustrations of a Wiegand inductor responding to a changing magnetic field in accordance with an embodiment of the present disclosure;

FIG. 7 is a hysteresis diagram of the Wiegand inductor of FIGS. 6A-6F;

FIG. 8 is a graphical illustration of electrical output of the Wiegand inductor of FIGS. 6A-6F;

FIG. 9 is a graph of voltage output of the Wiegand inductor of FIGS. 6A-6F;

FIG. 10 is a detail view of a first voltage pulse of the graph of FIG. 9;

FIG. 11 is a graphical illustration of electrical output of a dynamo in accordance with an embodiment of the present disclosure; and

FIG. 12 is a graph of voltage output of the dynamo of FIG. 11.

Referring to the drawings in which like reference characters refer to like parts throughout the several views thereof, the present disclosure is generally directed to a system and method for harvesting electrical energy from a rotating magnetic field generated by a blood pump of an extracorporeal circulation system, although the pump need not be a blood pump and the fluid may be a fluid other than blood and the system may be a fluid flow system.

### DETAILED DESCRIPTION

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. Spatial or directional terms, such as “left”, “right”, “inner”, “outer”, “above”, “below”, and the like, are not to be considered as limiting as the disclosed embodiments can assume various alternative orientations.

As used herein, the singular form of “a”, “an”, and “the” include plural referents unless the context clearly dictates otherwise.

All numbers used in the specification and claims are to be understood as being modified in all instances by the term “about”. The terms “approximately”, “about”, and “substantially” mean a range of plus or minus ten percent of the stated value.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

It is also to be understood that the specific devices and processes illustrated in the attached drawings, and described in the following specification, are simply exemplary examples of the disclosure. Hence, specific dimensions and other physical characteristics related to the examples disclosed herein are not to be considered as limiting.

The terms “first”, “second”, and the like are not intended to refer to any particular order or chronology, but refer to different conditions, properties, or elements.

The term “at least” is synonymous with “greater than or equal to”. The term “not greater than” is synonymous with “less than or equal to”.

As used herein, the term “dynamo” means an electrical component that generates an alternating (AC) output voltage in response to a changing magnetic field, where the magnitude of the output voltage has a positive correlation to the speed at which the magnetic field changes.

It is to be understood that the disclosure may assume alternative variations and step sequences, except where expressly specified to the contrary. It is also to be understood that the specific devices and processes illustrated in the attached drawings, and described in the following specification, are simply exemplary aspects of the disclosure. Hence, specific dimensions and other physical characteristics related to the examples disclosed herein are not to be considered as limiting.

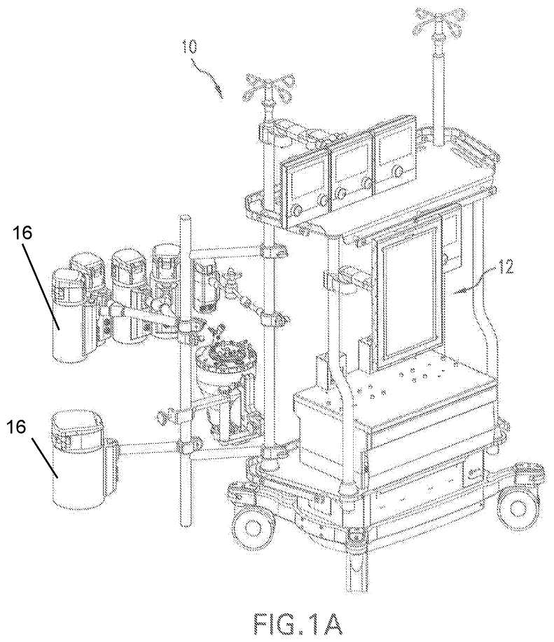

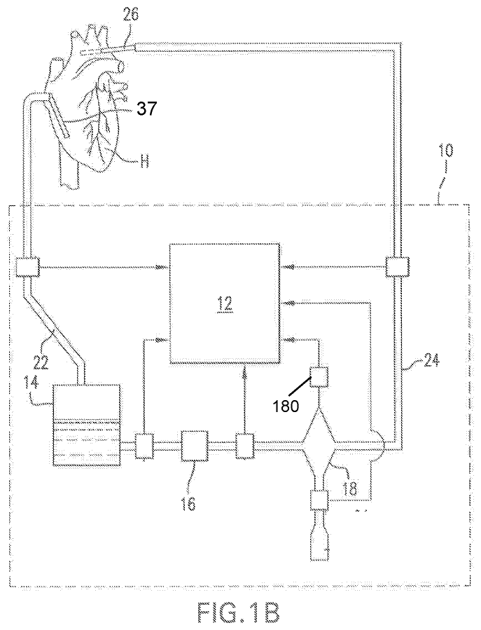

Referring first to FIGS. 1A and 1B, an extracorporeal circulation system 10 (also referred to as a heart-lung machine, or as a cardiac bypass system, or as a cardiopulmonary bypass system, and which should be construed broadly to include cardiopulmonary bypass (CPB) systems, minimal extracorporeal circulation (MECC) systems, extracorporeal membrane oxygenation (ECMO) systems (respiratory and cardiac), and pump assisted lung protection (PALP) systems) includes a controller 12, a venous reservoir 14, one or more blood pumps 16 and an oxygenator 18. The venous reservoir 14, blood pump 16 and oxygenator 18 are connected together in a conventional manner so that venous blood extracted from a cavoatrial cannula 37 inserted in the venous side of a patient's circulatory system, for example the vena cava and/or the right atrium of the heart H, flows via tubing 22 into the venous reservoir 14, and from there is pumped via the blood pump 16 into the oxygenator 18. The oxygenator 18 oxygenates the blood before the blood, now oxygenated, flows through tubing 24 and into an arterial cannula 26 inserted in the aortic root of the heart H. The blood pump 16 may be a roller pump or a centrifugal pump, each of which includes an electric motor in order to pump blood albeit via substantially different mechanisms as is known in the art. Commercial examples of suitable blood pumps 16 and associated components include, but are not limited to, Maquet Cardiohelp® and Maquet Rotaflow® systems offered by Maquet Cardiopulmonary GmbH. The extracorporeal circulation system 10 illustrated by FIGS. 1A and 1B constitutes a simplified, non-limiting illustration as such systems are generally much more complex.

With continued reference to FIG. 1B, at least one sensor 180 may be provided in association with the oxygenator 18 for measuring one or more parameters such as blood temperature, blood pressure, and blood flow rate. The at least one sensor 180 may be configured to detect or measure a property of blood flowing into, out of, or through the oxygenator 18, such as blood pressure, blood temperature, or flow rate. The at least one sensor 180 may further be configured to transmit an output data signal corresponding to the detected or measured property to the controller 12. The at least one sensor 180 may be provided at a location relative to the oxygenator 18 where clinically desirable, such as near an inlet of the oxygenator 18, an outlet of the oxygenator 18, or within the oxygenator 18. Such sensors 180 typically require a 5 volt power source in order to operate, although in some cased electronic sensors requiring only 1.2 volts, 1.8 volts or 3.3 volts may be available.

Further details of the extracorporeal circulation system 10 of the present disclosure is described in U.S. Provisional Patent Application No. 62/160,689, filed on May 13, 2015, and its corresponding U.S. Patent Application Publication No. US 2018/0344919, both of which are incorporated herein by reference in their entireties for all they disclose.

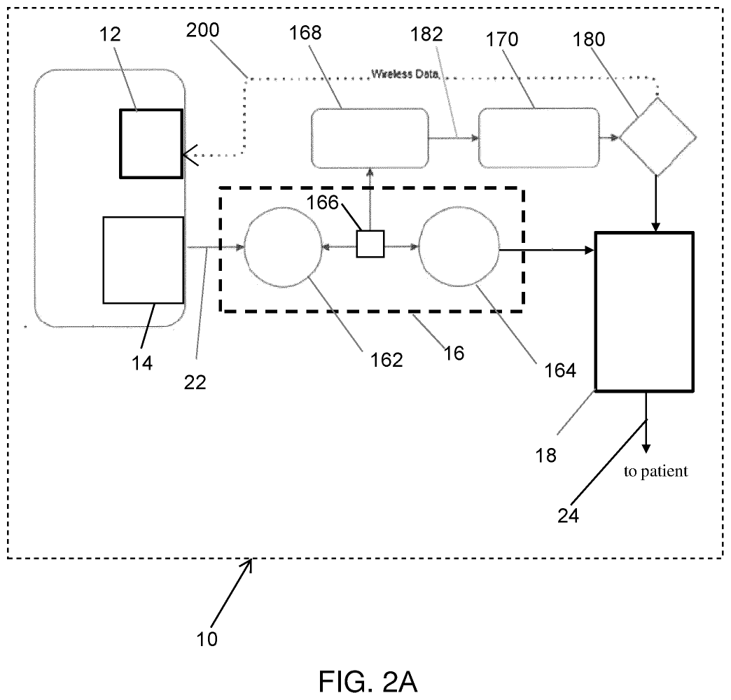

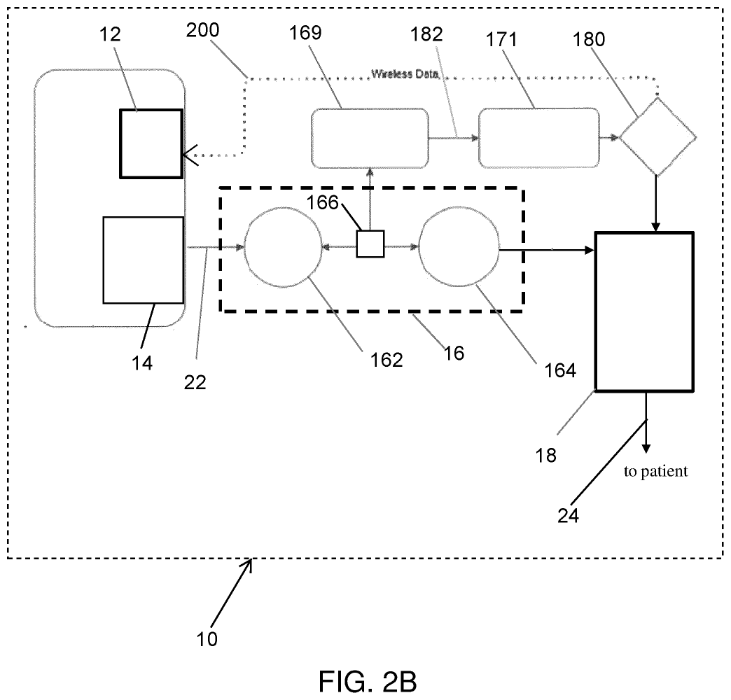

Referring now to FIGS. 2A and 2B, the extracorporeal circulation system 10 in accordance with the present disclosure includes components for harvesting energy from the electric motor of a blood pump 16 to provide electrical power to the at least one sensor 180 and/or other circuitry or electronic devices of the system 10. The blood pump 16 includes an electric motor 162, such as an induction motor, connected to and configured to drive a pump rotor 164 (e.g. an impeller). The motor 162 and the rotor 164 may be magnetically coupled by a magnetic coupling 166, namely, a coupling magnetic field, such that rotation of the rotor 164 by the motor 162 generates an alternating magnetic field. The alternating magnetic field may be, for example, a rotating magnetic field generated during operation of the electric motor 162, which results in rotation of the rotor 164. Referring specifically to the embodiment shown in FIG. 2A, a transducer 168, which includes a Wiegand inductor, is disposed in operative proximity to the rotor 164 so as to be present in the alternating magnetic field. The transducer 168 is configured to generate electrical energy in response to changes in the alternating magnetic field associated with the rotor 164. Thus, transducer 168 may be referred to in this disclosure as a “magnetic transducer” because it converts the energy of an alternating magnetic field into electrical energy usable to power one or more electronic devices, such as electronic sensors and other electronic devices.

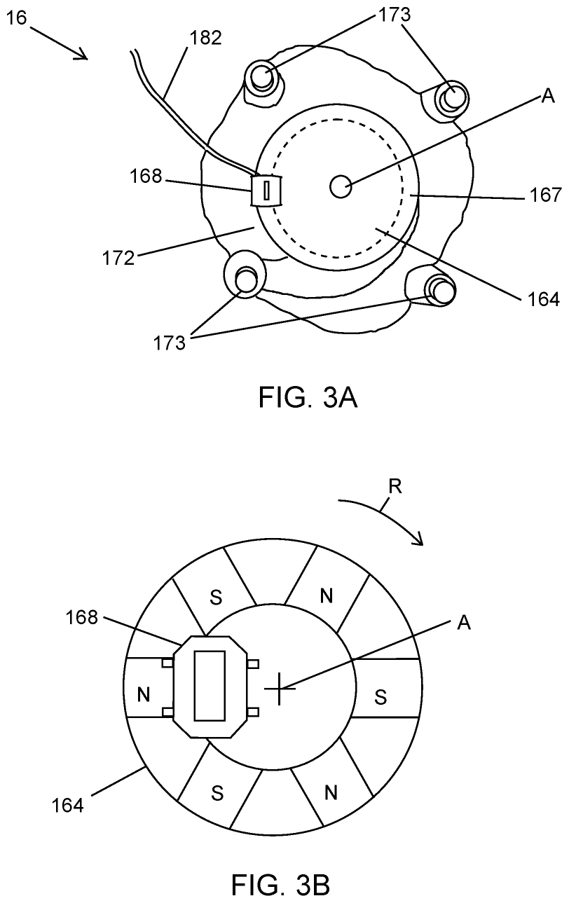

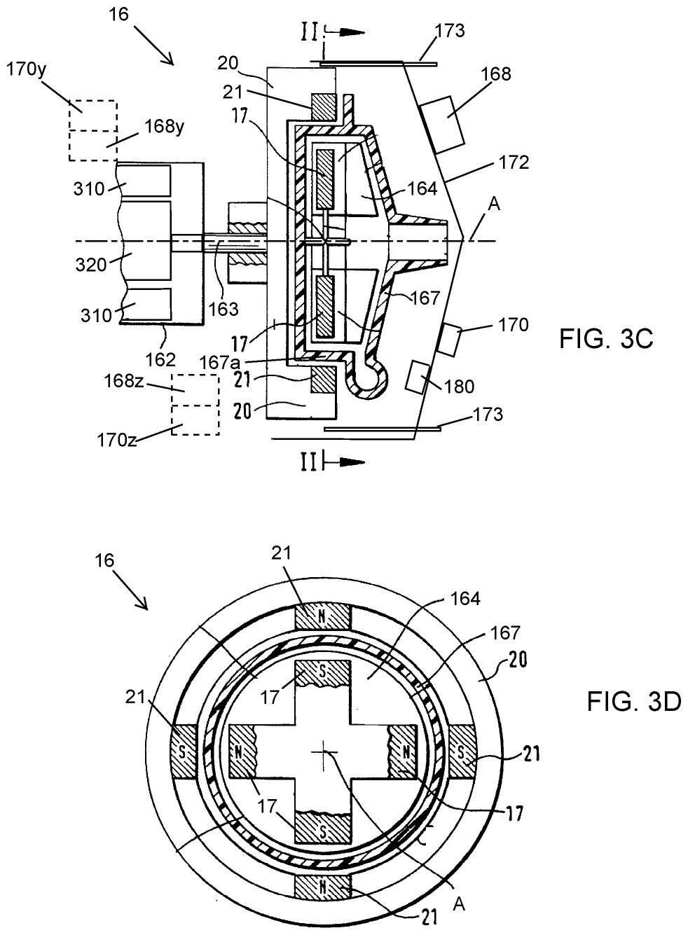

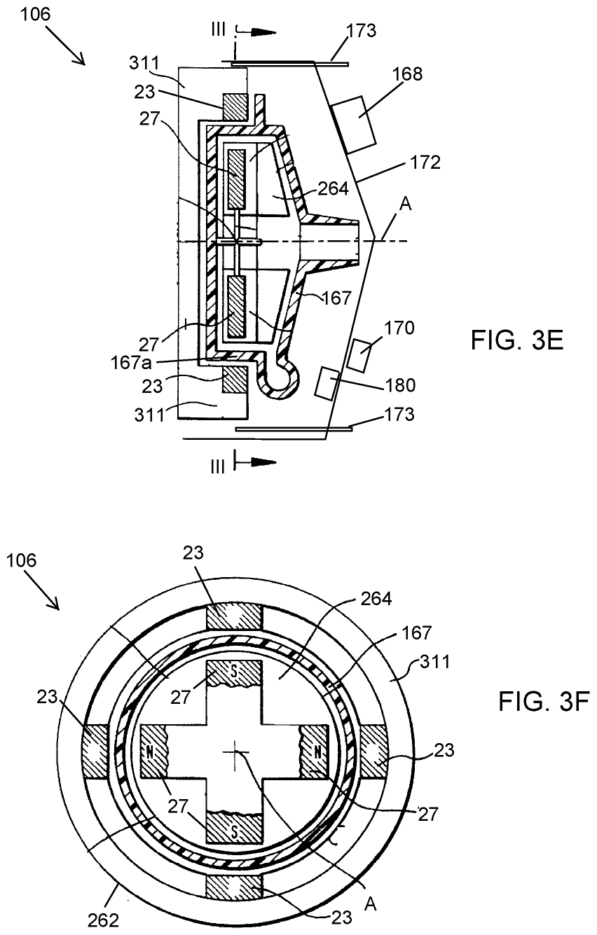

A non-limiting example of placement of the transducer 168 relative to the blood pump 16 is shown in FIGS. 3A-3D. In particular, the transducer 168 may be positioned in operative proximity to the rotor 164 and the motor 162 such that the transducer 168 is within an effective operable range of the alternating magnetic field generated during rotation of the rotor 164 so that the transducer 168 is able to convert energy from the changing magnetic field into usable electrical energy suitable for powering electronic devices. In some embodiments, as shown in FIG. 3A, the transducer 168 may be disposed on or in a housing 167 of the blood pump 16, such as a cover 172 of the housing 167 that is removable and reattachable to the blood pump 16 by one or more fasteners 173 to allow replacement of the rotor 164. The cover 172, or any other part of the housing 167 to which the transducer 168 is mounted, may be a non-magnetic material such as a plastic so as to not interfere with the alternating magnetic field associated with rotation of the rotor 164. As shown in FIG. 3A, the cover 172, or any other part of the housing 167 to which the transducer 168 is mounted, may be transparent to allow for visual inspection of the rotor 164.

As shown in FIGS. 3A-3B, the transducer 168 may be located off-center from a rotational axis A of the rotor 164. However, the position of the transducer 168 relative to the rotational axis A is not limited to the position shown FIG. 3A. Rather, the transducer 168 may be located at substantially any position with respect to the rotational axis A so long as the transducer 168 is present in the changing magnetic field at a location that permits the transducer 168 to harvest magnetic energy for conversion into electrical energy. FIG. 3B shows the transducer 168 overlaid with the rotor 164, including the alternating north poles N and south poles S of magnets 17 (e.g. permanent magnets or electromagnets) of the rotor 164 that generate an alternating magnetic field about the rotational axis A when an electrical current is applied to the motor 162. As current is applied to the motor 162 causing the rotor 164 to rotate in the direction R, the transducer 168 harvests energy generated by the alternating magnetic field.

Referring now to FIGS. 3C and 3D, in non-limiting embodiments, the magnets 17 may be attached to or embedded in the rotor 164, which is magnetically coupled to the motor 162 via a coupling plate 20. The electric motor 162 may include a motor stator 310 and a motor rotor 320 that operate to generate an alternating magnetic field in order to rotate the rotor 320 that is connected to shaft 163 in order to rotate the shaft 163 when an electrical current is applied to the motor 162. For the purpose of this disclosure, the alternating magnetic field associated with operation of the motor stator 310 and/or the motor rotor 320 may be referred to as the alternating magnetic field of the motor.

Various designs of the motor stator 310 and the motor rotor 320 are possible in line with conventional motor designs known in the art. For example, the motor rotor 320 may include a plurality of permanent magnets, and the stator motor 310 may include a plurality of electromagnets that are periodically changed in polarity to generate an alternating magnetic field that drives rotation of the magnets in the motor rotor 320. More particularly, the alternating magnetic field of the motor stator 310 may interact with a magnetic field of the motor rotor 320 to generate a torque that causes the motor rotor 320 to rotate relative to the stator 310. Other embodiments of the motor stator 310 and the motor rotor 320 will be readily appreciated by those skilled in the art as conventional motor designs, such as the motor rotor 320 including electromagnets and the motor stator 310 including permanent magnets, or both the motor stator 310 and the motor rotor 320 including electromagnets. The motor 162 may be powered by either AC or DC power using appropriate circuitry as is conventionally known in the art. Thus, electric motor 162 may be a direct current (DC) motor or an alternating current (AC) motor.

With continued reference to FIGS. 3C-3D, the shaft 163 extends from the motor rotor 320 and is coupled to the coupling plate 20, which includes a plurality of magnets 21 (e.g. permanent magnets or electromagnets). The magnets 21 of the coupling plate 20 may be circumferentially arranged to have alternating north poles N and south poles S. The number of magnets 21 of the coupling plate 20 may be the same as the number of magnets 17 of the pump rotor 164 so that the magnets 21 of the coupling plate 20 have a one-to-one relationship with the magnets 17 of the pump rotor 164. The magnets 21 of the coupling plate 20 may interact with the magnets of 17 of the pump rotor 164 to create a magnetic coupling 166 (labelled in FIGS. 2A-2B) between the coupling plate 20 and the pump rotor 164. In particular, the pump rotor 164 may tend to self-align when brought into proximity with the magnetic coupling plate 20 such that the magnets 17 of the pump rotor 164 having north N polarity tend to oppose the magnets 21 of the coupling plate 20 having south S polarity, and vice versa, as shown in FIG. 3D. As shown in FIG. 3C, portions 167a of the housing 167 may extend between the magnets 17 of the pump rotor 164 and the magnets 21 of the coupling plate 20 without interfering with the magnetic coupling 166 between the pump rotor 164 and the coupling plate 20. When the coupling plate 20 is rotated by the shaft 163, the pump rotor 164 rotates in the same direction as the shaft 163 and at substantially the same speed due to the magnetic coupling effect between the magnets 17 of the pump rotor 164 and the magnets 21 of the coupling plate 20. In other words, while the pump rotor 164 and the coupling plate 20 are not directly and securely affixed to one another by a mechanical connection, the magnetic coupling 166 existing between the magnets 17 of the pump rotor 164 and the magnets 21 of the coupling plate 20 serves as a magnetic connection that effects rotation of the pump rotor 164 when the coupling plate 20 is rotated by the shaft 163. The magnets 17, 21 of the pump rotor 164 and the coupling plate 20 generate an alternating (e.g. rotating) magnetic field as the coupling plate 20 and the pump rotor 164 are rotated by the motor 162. For the purposes of this disclosure, this alternating magnetic field associated with rotation of the pump rotor 164 and/or the coupling plate 20 may be collectively referred to as the alternating magnetic field of the pump rotor.

The alternating magnetic field of the motor and the alternating magnetic field of the pump rotor constitute substantially different alternating magnetic fields as each originates from a substantially different magnetic source. It is within the scope of the present disclosure to place one or more transducers 168, or one or more dynamos 169, or a combination of transducers 168 and dynamos 168, at one or more locations such that these devices harvest electrical energy from primarily the alternating magnetic field of the pump rotor, or primarily from the alternating magnetic field of the motor, or from substantially both the alternating magnetic field of the pump rotor and the alternating magnetic field of the motor.

With continued reference to FIG. 3C, in some embodiments, the transducer 168 may be positioned in proximity to the alternating (e.g. rotating) magnetic field generated by the motor stator 310 and/or motor rotor 320, i.e. the alternating magnetic fields induced by the periodic change in the polarity of the stator 310 and/or by rotation of the motor rotor 320, which are collectively referred to as the alternating magnetic field of the motor. The transducer 168 may thus harvest energy directly from the alternating magnetic field of the motor 162. Additionally or alternatively, the transducer 168 may be located in operative proximity to the pump rotor 164 to harvest energy from the alternating (e.g. rotating) magnetic field generated by rotation of the magnets 17, 21 of the pump rotor 164 and coupling plate 20. That is, the transducer 168 may harvest energy from either or both of an alternating magnetic field associated with the motor stator 310 and the motor rotor 320, and from an alternating magnetic field associated with the coupling plate 20 and the pump rotor 164.

In FIG. 3C, such alternate locations for transducer 168 and associated conversion box 170 are shown in phantom (identified by reference numerals 168y, 170y and 168z, 170z) as an integrated circuit or integrated electronic chip, although they could be separate and electrically connected by one or more wires or by a wireless electric connection. Likewise, while transducer 168 and conversion box 170 located on the cover 172 are shown disposed apart, such that one or more wires (not shown in FIG. 3C) or a wireless electric connection (not shown in FIG. 3C) connect them so that voltage pulses generated by the transducer 168 are sent to conversion box 170 for processing into a useable output voltage, such as in the range of 1.2 volts to 5.0 volts or higher, these separate structure may be integrated together into an integrated circuit or to form an integrated electronic chip.

Additional details of the blood pump 16 as shown in FIGS. 3A-3D are described in U.S. Pat. No. 5,658,136, issued on Aug. 19, 1997, the entirety of which is hereby incorporated by reference in its entirety.

FIGS. 3E-3F illustrates another non-limiting embodiment of the blood pump 106 in which the pump rotor 264 also serves as a motor rotor, in that the rotor 264 is directly driven by a stator of the motor 262; thus, in this embodiment shaft 163 and coupling plate 20 are not included. In this embodiment, the rotor 264 may be substantially the same as described in connection with FIGS. 3C-3D. The motor 262 includes a motor stator 311 including one or more magnets 23 (e.g. electromagnets) operatively associated with the magnets 27 of the rotor 264. During operation of the motor 262, which may be a DC motor or an AC motor, current is supplied to the electromagnets 23 to periodically change the polarity of the electromagnets 23. The alternating polarity of the electromagnets 23 of the stator 311 generates an alternating magnetic field that causes the rotor 264 to rotate about the axis A. More particularly, the alternating magnetic field of the motor stator 311 may interact with the magnetic field of the magnets 27 of the rotor 264 to generate a torque that causes the rotor 264 to rotate relative to the stator 311. The transducer 168 may be located in operative proximity to the stator 311 in order to harvest energy from the alternating magnetic field associated with the motor stator 311, i.e. the alternating magnetic field induced by the periodic change in the polarity of the magnets 23. Additionally or alternatively, the transducer 168 may be located in operative proximity to the rotor 264 to harvest energy from the alternating magnetic field associated with rotation of the magnets 27 of the rotor 264.

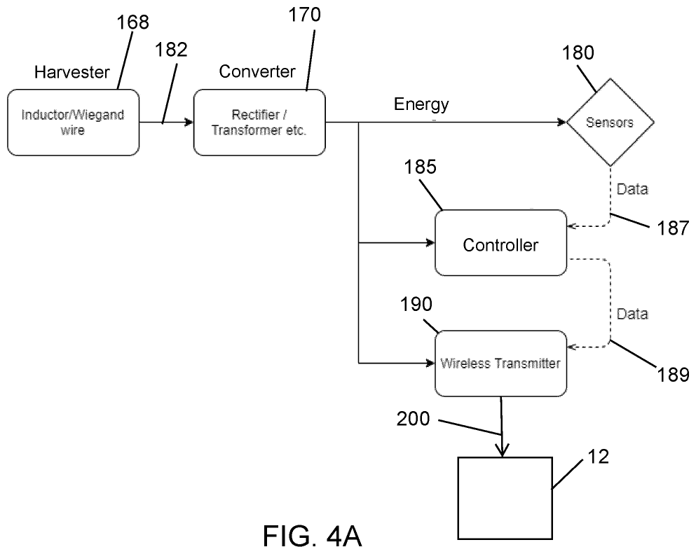

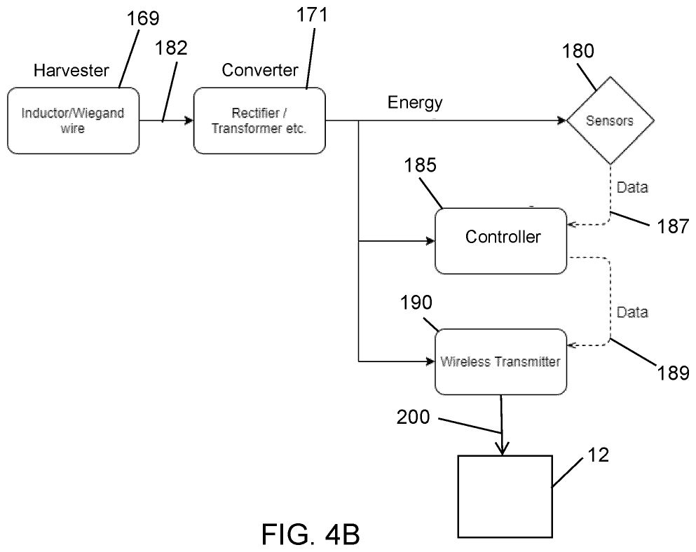

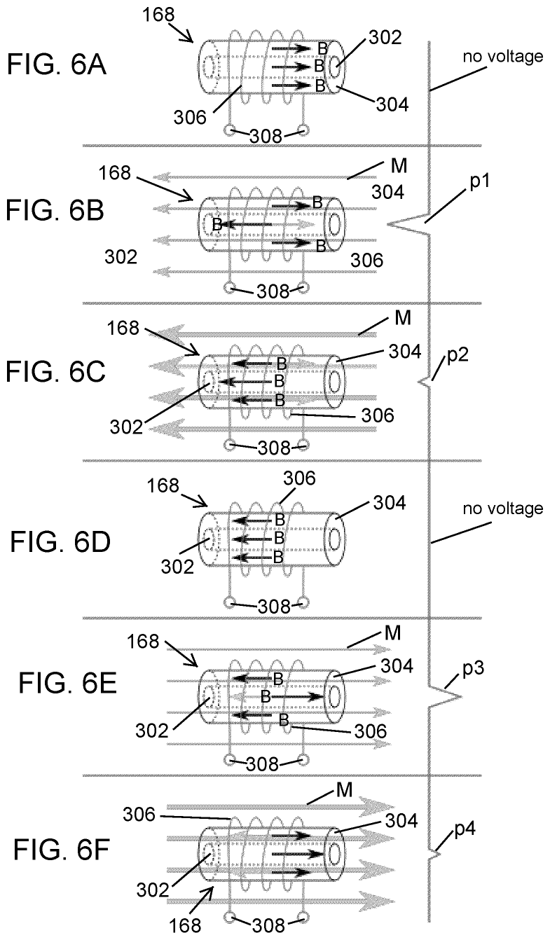

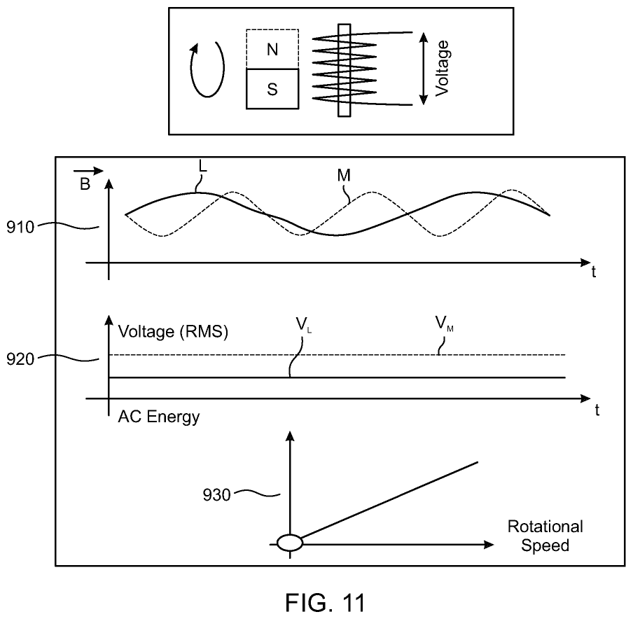

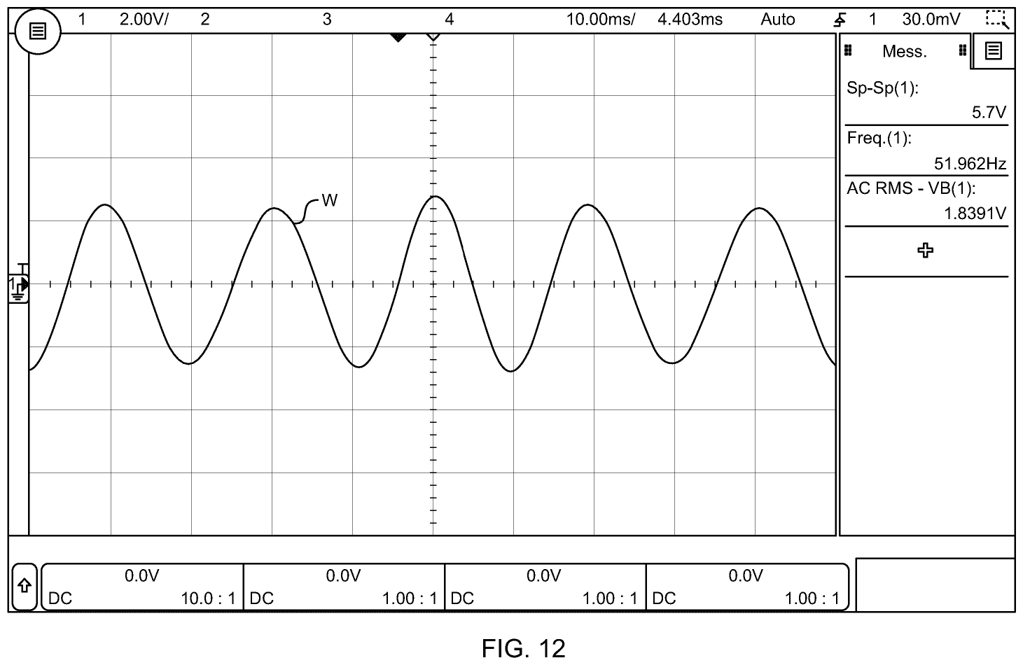

As indicated above, the transducer 168 includes a Wiegand inductor, which generates a series of voltage pulses of substantially uniform amplitude in response to rotation of the alternating magnetic field associated with the rotor 164, 264. In some embodiments, the transducer 168, in the form of a Wiegand inductor, may be replaced with a dynamo 169 (as shown in FIGS. 2B and 4B), which generates an AC voltage output proportional to rotational speed of the rotor 164, 264 in response to rotation of the alternating magnetic field associated with the rotor 164, 264. The dynamo 169 may be positioned within the alternating magnetic field associated with the rotor 164, 264 in the same manner as the transducer 168 shown in FIGS. 3A-3B. Additional details of the electrical properties and electrical outputs associated with the magnetic transducer 168, in the form of a Wiegand inductor, are described herein in connection with FIGS. 6A-10, and additional details of the electrical properties and electrical outputs associated with the dynamo 169 are described herein in connection with FIGS. 11 and 12.

It should be noted that a Wiegand sensor is a Wiegand inductor adapted to sense the number and timing of changes in a magnetic field. In accordance with this disclosure, a Wiegand inductor is a device that is used to harvest electrical energy from a changing magnetic field generated by, for example, an electric motor and/or a magnets of a pump rotor, in a novel, non-obvious, inventive way regardless of whether the Wiegand inductor is also used to sense the number and timing of changes in the changing magnetic field from which electrical energy is harvested. In accordance with an embodiment of this disclosure, the Wiegand inductor is adapted to harvest electrical energy from a changing magnetic field and this Wiegand inductor is not adapted to collect data on the number and timing of changes in the magnetic field so it is not construable as a Wiegand sensor.

In some embodiments of this disclosure, the transducer 168, 169 may be disposed to harvest electrical energy from primarily a changing magnetic field generated by an electric motor, or the transducer 168, 169 may be disposed to harvest electrical energy from primarily a changing magnetic field generated by magnets of a rotating rotor 164, 264, or the transducer 168, 169 may be disposed to harvest electrical energy substantially from both a changing magnetic field generated by an electric motor and a changing magnetic field generated by magnets of a rotating rotor 164, 264. In the embodiment(s) described with respect to FIGS. 3C-3D, when the transducer 168, 169 is disposed to harvest electrical energy from both the changing magnetic field generated by the motor 162 and the changing magnetic field generated by magnets of the rotating rotor 164, the two changing magnetic fields may effect the transducers 168 and 169 differently. It is noted that the two changing magnetic fields are changing in phase with one another because the motor rotor 320 and the pump rotor 164 are rotating at substantially the same rate and together. Consequently, with respect to the dynamo 169, more electrical energy is harvested per rotation cycle due to the cumulative effect of the combined changing magnetic fields.

With respect to transducer 168, more electrical energy is not necessarily harvested from the combined magnetic fields because the Wiegand inductor generates only pulses of a fixed amplitude so long as a threshold is reached that causes polarity shifts as will be described below with respect to FIGS. 6A-6F. However, the combined magnetic fields are in phase so they may not trigger voltage pulses more frequently than when harvesting electrical energy from one of the single sources of a changing magnetic field. On the other hand, the cumulative effect of the combined changing magnetic fields is that they permit an expanded region in which the transducer 168 may be placed to harvest electrical energy from the combined changing magnetic fields.

When operating multiple pumps 16 in a pump array, as shown in FIG. 1A, it is possible to dispose a transducer 168 so it harvests electrical energy from two neighboring pumps 16, which may not be operating in phase. In this case, the transducer 168 may be triggered by the combined magnetic fields to generate voltage pulses at a frequency greater than the frequency of the changing magnetic fields of either of the two neighboring pumps 16. Thus, by judicious selection of where to place a transducer 168 so it is exposed to a combined changing magnetic field from two or more sources of changing magnetic fields that are not in phase, it becomes possible to harvest more power from the combined magnetic fields using the transducer 168 because the faster transducer 168 generates voltage pulses the more electrical power it is harvesting from the combined changing magnetic fields.

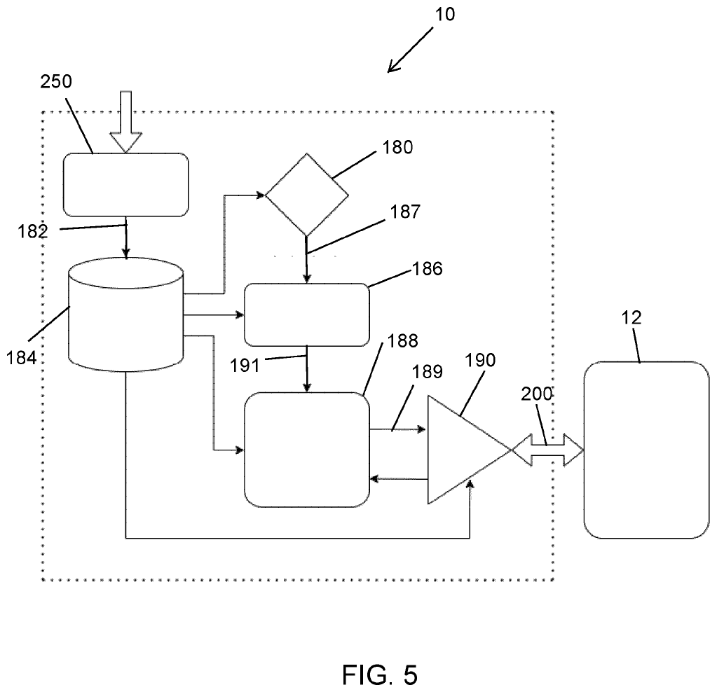

Referring back to FIG. 2A, the electrical energy generated by the transducer 168 may be used to power the at least one sensor 180 operatively associated with the oxygenator 18. The transducer 168 may be in electronic communication with the at least one sensor 180 via a power lead 182 that supplies electrical energy from the transducer 168 to the at least one sensor 180. The at least one sensor 180 may constitute, for example, a blood pressure sensor, a blood temperature sensor, a flow rate sensor, a distance sensor, or the like. The at least one sensor 180 may be configured to detect or measure a property of blood flowing into, out of, or through the oxygenator 18, such as blood pressure, blood temperature, or flow rate. The at least one sensor 180 may be further configured to transmit, either directly or indirectly, an output data signal corresponding to the detected or measured property to the controller 12. In some embodiments, as discussed herein in connection with FIGS. 4 and 5, additional circuitry associated with the at least one sensor 180 may receive the output data signal from the at least one sensor 180 and transmit the output data signal to the controller 12. The controller 12 may be programmed or configured to receive the output data signal from the at least one sensor 180, or from the additional circuitry associated with the at least one sensor 180, and, based on the received output data signal, display data on a GUI display associated with operation of the system 10 and/or adjust operation of the system 10.

Because the at least one sensor 180 is powered by the transducer 168, which itself is disposed on or in proximity to the blood pump 16, neither a power cable from the controller 12 nor another external power source (e.g. a battery) is required to power the at least one sensor 180. As such, setup time of the system 10 is reduced, and the potential for making an improper power connection to the at least one sensor 180 is eliminated.

In some embodiments, the at least one sensor 180 may be configured to wirelessly transmit the output data signal to the controller 12. Thus, a signal cable need not be provided between the at least one sensor 180 and the controller 12. As such, setup time of the system 10 is reduced, and the potential for making an improper signal connection between the controller 12 and the at least one sensor 180 is eliminated. The wireless output data signal, indicated by reference numeral 200 in FIG. 2A, may be transmitted by any conventional wireless protocol such as Wi-Fi, nearfield communication (NFC), Bluetooth®, or the like.

With continued reference to FIG. 2A, a conversion box 170 may be provided between the transducer 168 and the at least one sensor 180 to store and condition the electrical energy generated by the transducer 168. The conversion box 170 includes circuitry to convert the electrical energy output by the transducer 168 into a power form usable by the at least one sensor 180. For example, the conversion box 170 may include circuitry to limit the voltage received by the at least one sensor 180 to prevent damage to the at least one sensor 180. The specific circuitry provided in the conversion box 170 may vary depending on the specifications of the transducer 168 and on the specifications of the at least one sensor 180. For example, in embodiments in which the transducer 168 is a Wiegand inductor, the conversion box 170 may include at least one rectifier to rectify voltage pulses generated by the transducer 168, and at least one capacitor for storing the rectified pulses of energy generated by the transducer 168 and outputting the stored energy to the at least one sensor 180 (or other circuit components) as a power output voltage. The conversion box 170 may further include a protection circuit for limiting the output voltage to a predetermined threshold suitable for safely operating the at least one sensor 180. In some embodiments, the conversion box 170 may output a DC voltage of between approximately 1.2 volts and approximately 5.0 volts, or between approximately 1.8 volts and approximately 5.0 volts, or between approximately 3.3 volts and approximately 5.0 volts.

Referring again to FIGS. 3C and 3E, in some embodiments, the transducer 168, the conversion box 170, and the at least one sensor 180 may all be disposed on a portion of the housing 167, e.g. the cover 172, of the blood pump 16. As such, the rotor 164, housing 167, cover 172, transducer 168, conversion box 170, and at least one sensor 180 may form a module (i.e. a disposable module) that can be readily replaced as a single unit at regular maintenance intervals consistent with predetermined hygienic practices. This is possible because, as described herein, the transducer 168 eliminates the need for external power connections supplying the conversion box 170 and the at least one sensor 180. Further, the wireless data transfer of the at least one sensor 180 eliminates the need for a signal cable between the at least one sensor 180 and the controller 12 of the hear-lung machine. As such, the health care provider does not need to perform any electrical disconnection in order to remove the transducer 168, conversion box 170, and at least one sensor 180 from the blood pump 16, in contrast to conventional systems. Provision of the rotor 164, housing 167, cover 172, transducer 168, conversion box 170, and at least one sensor 180 as a single, modular unit reduces the likelihood and severity of operator error that may be associated with replacing these components individually.

Referring now to FIG. 2B, another embodiment of the extracorporeal circulation system 10 in accordance with the present disclosure is shown. The embodiment of FIG. 2B is substantially similar to the embodiment of FIG. 2A, and only the differences will be described. The embodiment of FIG. 2B utilizes a transducer 169 in the form of a dynamo in contrast to the Wiegand inductor transducer 168 of FIG. 2A. The dynamo 169 of FIG. 2B may generate an AC voltage output proportional to rotational speed of the rotor 164 in response to rotation of the alternating magnetic field associated with the rotor 164. Additional details of the electrical properties and electrical outputs associated with the various forms of magnetic transducer 169, in the form of a dynamo, are described herein in connection with FIGS. 11-12.

Because the output of the dynamo 169 differs from the Wiegand sensor 168 of FIG. 2A, the conversion box 170 of FIG. 2A is replaced with conversion box 171 in the embodiment of FIG. 2B. The conversion box 171 may include one or more inductors for accumulating AC energy generated by the dynamo 169; one or more rectifiers for rectifying voltage flowing into or out of the one or more inductors; and one or more components (e.g. a circuit or subcircuit) for converting AC voltage generated by the dynamo 169 to DC voltage suitable for powering one or more electronic components, such as the at least one sensor 180. Such components for converting AC voltage to DC voltage, which may include for example rectification of the AC voltage, may constitute conventional, commercially available units. In some embodiments, the conversion box 171 may output a DC voltage of between approximately 1.2 volts and approximately 5.0 volts, or between approximately 1.8 volts and approximately 5.0 volts, or between approximately 3.3 volts and approximately 5.0 volts.

FIGS. 2A and 2B illustrate non-limiting exemplary circuit embodiments for powering the at least one sensor 180 via the transducer 168, 169; however, the system 10 may include additional circuitry, as shown in FIGS. 4A, 4B, and 5, to facilitate and/or improve performance of the transducer 168, 169 and/or the at least one sensor 180. Referring to FIG. 4A, the output of the transducer 168 may be in electronic communication with the conversion box 170 in substantially the manner shown in FIG. 2A. As described herein in connection with FIG. 2A, the conversion box 170 converts the pulsed electrical energy output from the transducer 168 to a suitable form (e.g. a suitable DC voltage) for powering the at least one sensor 180. Furthermore, the conversion box 170 may convert the pulsed electrical energy output from the transducer 168 to a suitable form (e.g. a suitable DC voltage) for powering various other electrical components, such as at least one sensor processor 185 and at least one transmitter 190. In particular, the conversion box 170 may output a DC voltage of between approximately 1.2 volts and approximately 5.0 volts, or between approximately 1.8 volts and approximately 5.0 volts, or between approximately 3.3 volts and approximately 5.0 volts.

The at least one sensor processor 185 may be in electronic communication with the at least one sensor 180 via a wired or wireless data connection 187. The sensor processor 185 may be configured to receive the output data signal (e.g. an analog output signal) from the at least one sensor 180 via the data connection 187 and convert that output data signal to a sensor data signal (e.g., a digital signal) for transmission to the at least one transmitter 190. The sensor processor 185 may include a processor, as is conventionally known in the art, configured to execute an algorithm for converting the output data signal from the sensor 180 to the sensor data signal outputted from the sensor processor 185. The transmitter 190 may be in electronic communication with the at least one sensor processor 185 via a wired or wireless data connection 189. The at least one transmitter 190 may be configured to transmit the sensor data signal to the controller 12 of the heart-lung machine.

In some embodiments, the at least one transmitter 190 may wirelessly transmit the sensor data signal to the controller 12. Thus, a signal cable need not be provided between the at least transmitter 190 and the controller 12. As such, setup time of the system 10 is reduced, and the potential for making an improper signal connection between the controller 12 and the at least one transmitter 190 is eliminated. The at least one transmitter 190 may wirelessly output the data signal 200 using any conventional wireless protocol such as Wi-Fi, nearfield communication (NFC), Bluetooth®, or the like.

FIG. 4B illustrates another non-limiting exemplary circuit embodiment substantially the same as FIG. 4A, with the exception that the Wiegand inductor 168 and associated conversion box 170 are replaced with the dynamo 169 and associated conversion box 171 as described herein in connection with FIG. 2B. As such, the embodiment shown in FIG. 4B harvests energy from an alternating magnetic field using the dynamo 169 to provide electrical power to one or more of the at least one sensor 180, the at least one sensor processor 185, and the at least one transmitter 190. The conversion box 171 converts the AC electrical energy output from the transducer 169 to a suitable form (e.g. a suitable DC voltage) for powering one or more of the at least one sensor 180, the at least one sensor processor 185, and the at least one transmitter 190. In particular, the conversion box 171 may output a DC voltage of between approximately 1.2 volts and approximately 5.0 volts, or between approximately 1.8 volts and approximately 5.0 volts, or between approximately 3.3 volts and approximately 5.0 volts. The at least one sensor 180, the at least one sensor processor 185, and the at least one transmitter 190 may be identical in form and function to those described in connection with FIG. 4A.

FIGS. 4A and 4B shows the at least one sensor 180, the at least one sensor processor 185, and the at least one transmitter 190 as discrete components for the clarity of illustration. However, it will be understood by those skilled in the art that any or all of these components may be integrated into a single physical device without deviating from the scope of the present disclosure, and which is powered by electrical energy harvested from an alternating magnetic field using at least one magnetic transducer 168 or dynamo 169. Of course, it is within the scope of this disclosure to employ a plurality of magnetic transducers 168 or a plurality of dynamos 169, or a mix thereof, to harvest electrical energy from an alternating magnetic field generated by a pump motor or other electric motor in order to provide electrical power to energize the single physical device, which may be an integrated circuit or computer chip.

Referring now to FIG. 5, another embodiment of circuitry associated with the transducer 168, 169, in the form of either a Wiegand inductor or dynamo respectively, and the at least one sensor 180 is illustrated in which the additional circuitry facilitates and/or improves performance of the transducer 168, 169 and/or the at least one sensor 180.

The circuit of FIG. 5 includes an energy harvester 250 that may include, in a first embodiment, the combination of the Wiegand inductor 168 and its associated conversion box 170 or, in a second embodiment, the combination of the dynamo 169 and its associated conversion box 171. In either embodiment, the energy harvester 250 constitutes a power source in the form of a DC voltage suitable for powering one or more electronic components as shown in FIG. 5. An output of the energy harvester 250 may be in electrical communication with an energy storage component 184. The energy storage component 184 may receive electrical energy generated by the energy harvester 250, and the energy storage component 184 may supply electrical energy in suitable form (e.g. a suitable DC voltage) to power one or more electrical components. In particular, the energy storage component 184 may output a DC voltage of between approximately 1.2 volts and approximately 5.0 volts, or between approximately 1.8 volts and approximately 5.0 volts, or between approximately 3.3 volts and approximately 5.0 volts. One or more outlets of the energy storage component 184 may be in electrical communication with, and may supply power within the aforementioned ranges of voltages to, one or more of the at least one sensor 180, the at least one signal processer 186 associated with the at least one sensor 180, the at least one data processor 188, and the at least one transmitter 190. Each of these components will be described in turn. The at least one signal processor 186 may be in electronic communication with the at least one sensor 180 via the wired or wireless data connection 187. The at least one signal processor 186 may be configured to receive the output data signal (e.g. an analog output signal) from the at least one sensor 180 via the data connection 187 and convert that output data signal to a sensor data signal (e.g., a digital signal) for transmission to the at least one data processor 188. The at least one signal processor 186 may include a signal processing unit, as is conventionally known in the art, configured to execute an algorithm for converting the output data signal to the sensor data signal.

The at least one data processor 188 may be in electronic communication with the at least one signal processor 186 via a wired or wireless data connection 191. The at least one data processor 188 may be configured to receive the sensor data signal from the at least one signal processor 186 and transmit the sensor data signal to the at least one transmitter 190. In some embodiments, the at least one data processor 188 may be configured to filter or otherwise convert the sensor data signal in a suitable manner prior to transmitting the sensor data signal to the at least one transmitter 190. In addition, the at least one data processor 188 may be configured to receive a verification signal from the at least one transmitter 190. The verification signal may include, for example, a handshake or checksum communication for verifying the authenticity of data sent between the at least one data processor 188 and the at least one transmitter 190. The at least one data processor 188 may include a microprocessor, as is conventionally known in the art, configured to execute an algorithm for transmitting the sensor data signal, and for receiving the verification signal from the at least one transmitter 190.

The at least one transmitter 190 may be in electronic communication with the at least one data processor 188 via a wired or wireless data connection 189. The at least one transmitter 190 may be configured to transmit the sensor data signal to the controller 12 of the heart-lung machine. In addition, the at least one transmitter 190 may be configured to receive a verification signal from the controller 12. The verification signal may include, for example, a handshake or checksum communication for verifying the authenticity of data sent between the at least one transmitter 190 and the controller 12. In some embodiments, the at least one transmitter 190 may be configured to communicate a sensor failure signal to the controller 12 and/or to communicate signal orders from the controller 12 to the at least one data processor 188.

In some embodiments, the at least one transmitter 190 may wirelessly transmit the sensor data signal to the controller 12. Thus, a signal cable need not be provided between the at least transmitter 190 and the controller 12. As such, setup time of the system 10 is reduced, and the potential for making an improper signal connection between the controller 12 and the at least one transmitter 190 is eliminated. The at least one transmitter 190 may wirelessly output the data signal 200 using any conventional wireless protocol such as Wi-Fi, nearfield communication (NFC), Bluetooth®, or the like.

FIG. 5 shows the at least one sensor 180, the energy storage component 184, the at least one signal processor 186, the at least one data processor 188, and the at least one transmitter 190 as discrete components for clarity of illustration. However, it will be understood by those skilled in the art that any or all of these components may be integrated into a single physical device, such as an integrated computer chip, without deviating from the scope of the present disclosure, and which is powered by electrical energy harvested from an alternating magnetic field using at least one magnetic transducer 168 or dynamo 169. Of course, it is within the scope of this disclosure to employ a plurality of magnetic transducers 168 or a plurality of dynamos 169, or a mix thereof, to harvest electrical energy from an alternating magnetic field generated by a pump motor or other electric motor in order to provide electrical power to the single physical device, which could be an integrated computer chip.

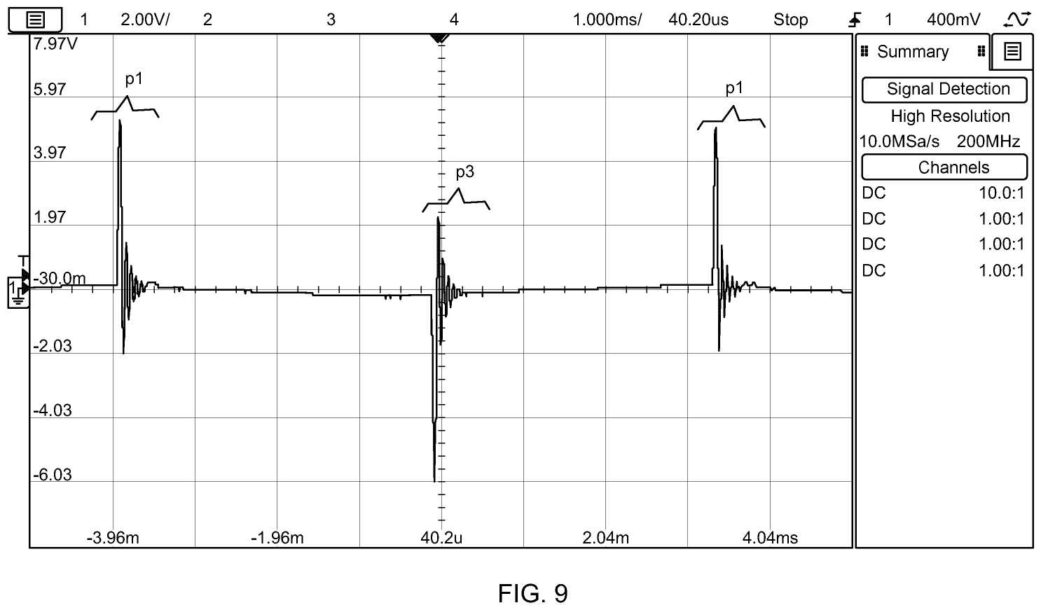

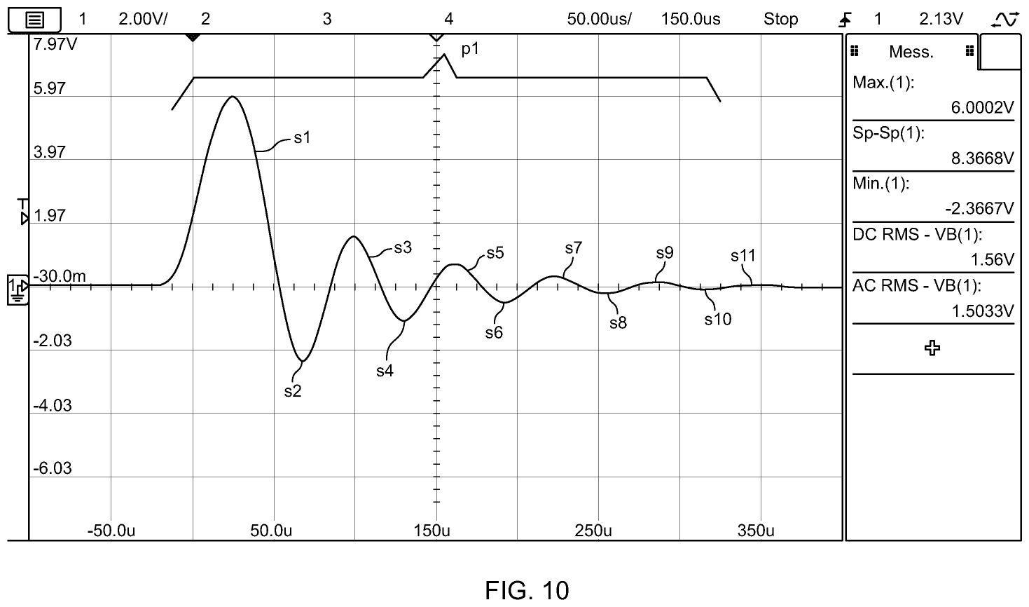

Referring now to FIGS. 6A-6F, the principle of energy harvesting and energy output of the transducer 168, in the form of a Wiegand inductor, is illustrated. It should be noted that FIGS. 6A-6F illustrate a reasonable theoretical model for explaining the voltage producing characteristics of a Wiegand inductor; however, this model may not explain all of the characteristics of voltage production empirically measured as evident from FIGS. 9 and 10.

In accordance with the theoretical model illustrated by FIGS. 6A-6F, the Wiegand inductor includes a Wiegand wire having an inner core 302 and an outer shell 304. The outer shell 304 has a higher magnetic coercivity than the inner core 302, such that the outer shell 304 requires exposure to a relatively stronger magnetic field to induce a change in polarity. The Wiegand wire may be composed of a low-carbon Vicalloy (e.g. a ferromagnetic alloy of cobalt, iron, and vanadium) that is subjected to a series of twisting and untwisting operations to cold-work the outer shell 304. This cold-working causes the outer shell 304 to become magnetically hard, while the inner core 302 remains magnetically soft. Following this cold-working, the Wiegand wire may be aged. Particular examples of suitable Vicalloy for the Wiegand wire includes an alloy of substantially 52% cobalt, 10% vanadium, trace amounts of elements such as carbon and manganese, and a balance (˜37%) of iron. A pickup coil 306 is wrapped around the Wiegand wire and has two terminals 308 between which a voltage is generated. Such voltage is generated between the terminals 308 when the Wiegand inductor is exposed to a changing magnetic field. More particularly, a first voltage pulse p1 is generated between the terminals 308 when the Wiegand inductor is exposed to a magnetic field of sufficient magnitude to reverse polarity of the inner core 302, and a second voltage pulse p2 is generated between the terminals 308 when the Wiegand inductor is subsequently exposed to a magnetic field of sufficient magnitude to reverse polarity of the outer shell 304.

Referring now to FIG. 6A, the transducer 168 is shown in the absence of an external magnetic field, with the respective polarities of both the inner core 302 and the outer shell 304 aligned in the same direction as indicated by the arrows B. In the state shown in FIG. 6A, no voltage is generated by the pickup coil 306. Referring next to FIG. 6B, the transducer 168 is shown exposed to an external magnetic field M of sufficient magnitude to reverse the polarity of the inner core 302, but insufficient magnitude to reverse the polarity of the outer shell 304. The switch in polarity of the inner core 302 generates the first voltage pulse p1 between the terminals 308 of the pickup coil 306. Referring next to FIG. 6C, the external magnetic field M is increased in magnitude relative to FIG. 6B, such that the magnetic field M has sufficient magnitude to reverse the polarity of the outer shell 304. The switch in polarity of the outer shell 304 generates the second voltage pulse p2 between the terminals 308 of the pickup coil 306. The polarity of the inner core 302 and the outer shell 304 is again aligned. The first and second voltage pulses p1, p2 may have substantially different magnitudes, although they possess the same polarity. In particular, in some cases the magnitude of second voltage pulse p2 may be negligible in energy output relative to first voltage pulse p1, such that no appreciable energy is harvested from second voltage pulse p2.

Referring now to FIG. 6D, the external magnetic field M is removed or at least reduced to zero, but the polarity of the inner core 302 and the outer shell 304 remains in the same state as is FIG. 6C. No voltage is generated by the coil 306. Referring now to FIG. 6E, the external magnetic field M is present in similar magnitude but in the opposite direction relative to FIG. 6B. The external magnetic field M is of sufficient magnitude to reverse the polarity of the inner core 302, but insufficient magnitude to reverse the polarity of the outer shell 304. The switch in polarity of the inner core 302 generates a third voltage pulse p3 between the terminals 308 of the pickup coil 306. Referring next to FIG. 6F, the external magnetic field M is increased in magnitude relative to FIG. 6E, such that the external magnetic field M has sufficient magnitude to reverse the polarity of the outer shell 304. The switch in polarity of the outer shell 304 generates a fourth voltage pulse p4 between the terminals 308 of the pickup coil 306. The polarity of the inner core 302 and the outer shell 304 is again aligned. When the magnetic field M is removed, or at least reduced to zero, the Wiegand inductor is again in the state of FIG. 6A, and the cycle of changing the external magnetic field M can be repeated.

The third and fourth voltage pulses p3, p4 may have substantially different magnitudes, although they possess the same polarity. In particular, in some cases the magnitude of fourth voltage pulse p4 may be negligible in energy output relative to third voltage pulse p3, such that no appreciable energy is harvested from fourth voltage pulse p4. The polarity of the third and fourth voltage pulses p3, p4 is opposite the polarity of first and second voltage pulses p1, p2. The magnitude of voltage pulses p1 and p3 are substantially the same, although they have opposite polarity. The magnitude of voltage pulses p2 and p4 are substantially the same, although they have opposite polarity.

The alternating magnetic field M of varying magnitude and direction as shown in FIGS. 6A-6F is generated by rotation of the rotor 164 of the blood pump 16 (see FIGS. 2A, 3A, and 3B). That is, as the rotor 164 rotates, an alternating magnetic field is generated which exposes the transducer 168 to cyclical changes in magnitude and direction of the magnetic field M as shown in FIGS. 6A-6F. The terminals 308 of the pickup coil 306 are attached to the power lead 182 (see FIG. 2A) such that mainly it is the first and third voltage pulses p1, p3 that are harvested and stored in the conversion box 170 and/or the energy storage component 184. This is because the magnitudes of the second and fourth voltage pulses p2, p4 are negligible compared to the first and third voltage pulses p1, p3, so the second and fourth voltage pulses p2, p4 have no appreciable contribution to the amount of energy harvested and stored.

The number of voltage pulses p1, p2, p3, p4 generated per unit time increases with the speed at which the magnetic field M alternates, meaning that the faster the rotor 164 is spun, the more energy is harvested per unit of time by the transducer 168 because the generation of voltage pulses p1, p2, p3, p4 occurs at a faster rate. However, the magnitude and polarity of voltage pulses p1 remain constant, and the magnitude and polarity of voltage pulses p2 remain constant, and the magnitude and polarity of voltage pulses p3 remain constant, and the magnitude and polarity of voltage pulses p4 remain constant. As will be appreciated from FIG. 9, each of the voltage pulses p1, p2, p3, p4 manifest as a series of oscillations. However, for ease of representation and explanation in FIGS. 6A-6F each of the voltage pulses p1, p2, p3, p4 is shown as a single spike in voltage. For the purposes of this disclosure, this pattern of voltage pulse generation of voltage pulses p1, p2, p3, p4 as shown by FIGS. 6A-6F, and which is the result of exposure of a Wiegand inductor to an alternating magnetic field (such as the rotating magnetic field generated by rotation of rotor 164), is referred to as the “Wiegand inductor voltage pulse generation pattern.”

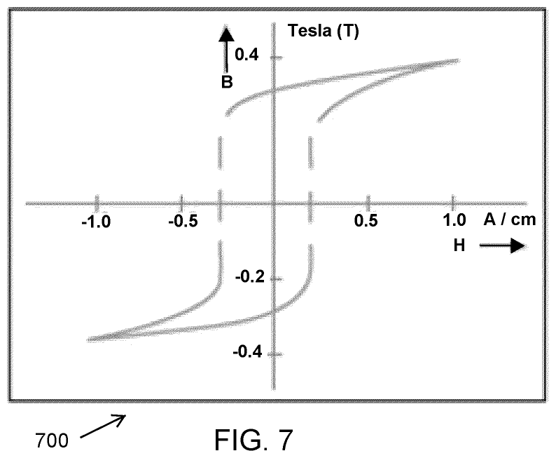

Referring now to FIG. 7, a magnetic hysteresis diagram 700 of the Wiegand wire (i.e., the inner core 302 and the outer shell 304) of FIGS. 6A-6B is illustrated in accordance with a non-limiting embodiment of this disclosure. In the diagram 700, magnetic field H in units of ampere per centimeter (A/cm) is plotted on the x-axis, and flux density B of the Wiegand wire in units of teslas is plotted on the y-axis. As may be appreciated from the diagram 700, the Wiegand wire has a high coercivity, or ability to withstand an external magnetic field without becoming demagnetized, which facilitates the generation of the voltage pulses p1, p2, p3, p4 described in connection with FIGS. 6A-6F.

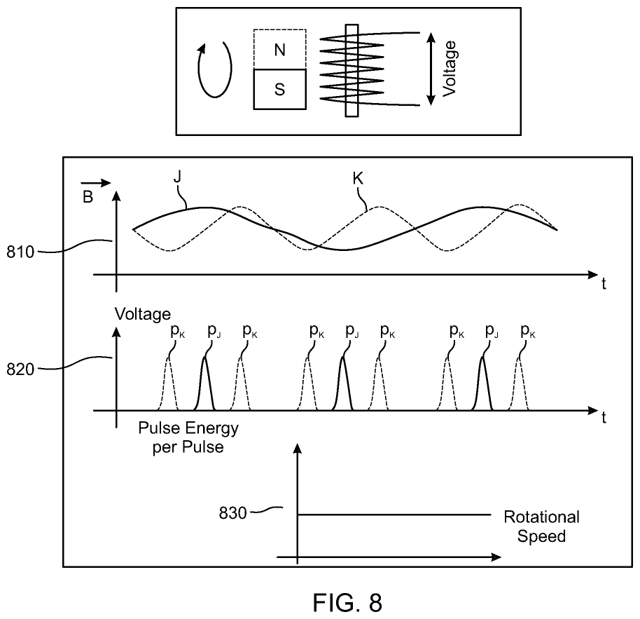

FIG. 8 provides a graphical illustration of the pulsed voltage output of the transducer 168 in the form of the Wiegand inductor of FIGS. 6A-6F. Graph 810 shows magnetic flux B plotted against time t. Graph 820 shows voltage V plotted against time t. Two different curves J and K of magnetic flux, illustrating two different rates of change of an alternating magnetic field, are represented in Graph 810. Curve J has a relatively slower change in flux over time in comparison to curve K. Graph 820 illustrates voltage pulse generation over time in relation to magnetic field changes of curves K and J represented in graph 810. As described herein in connection with FIGS. 6A-6F, voltage is generated by the Wiegand inductor in pulses p as the alternating magnetic field causes changes in polarity of the inner core 302 and the outer shell 304. The time interval at which the pulses p occur is a function of speed of the change in the magnetic field, in this case the speed of rotation of the rotor 164. Thus, a voltage pulse p_{J} associated with curve J occurs at each change in polarity of curve J, and a voltage pulse p_{K} associated with curve K occurs at each change in polarity of curve K. Curve K thus generates voltage pulses p_{K} at a faster rate than curve J generates voltage pules p_{J} because, as shown in graph 810, curve K has a faster rate of change in magnetic flux compared to curve J. It is noted, however, that the voltage pulses p_{K} associated with curve K have the same magnitude of the voltage pulses p_{J} associated with curve J because the magnitude of the pulse generated by the Wiegand inductor is independent of the rate of change of the alternating magnetic field.

Graphs 810 and 820 can be used to conceptualize how a transducer 168 may generate voltage pulses when placed within two rotating magnetic fields (i.e., generated from different magnetic sources such as two adjacent electric motors 162 of two neighboring pumps 16 of a pump array) at the same time that have substantially different rotating frequencies. A transducer 168 located to harvest electrical energy from magnetic fields represented by both curves J and K, which are out of phase, will produce voltage pulses p_{J} and p_{K} so the number of pulses generated over time will be the sum of voltage pulses p_{J} and p_{K}; thus, the transducer 168 will generate more pulses per unit time in the combined field than when it is located in just one changing magnetic field J or K. This equates to a greater generation of voltage power in the combined magnetic field. On the other hand, if the transducer 168 is placed within two changing magnetic fields that are in phase, for example, such as the rotating magnetic field J generated by an electric motor 162 and the rotating magnetic field J′ generated by magnets of its associated pump rotor 164, the number of voltage pulses p_{J} generated will be the same as if the transducer 168 was located in only a single rotating magnetic field because transducer 168 generates voltages of fixed pulse amplitude when experiencing polarity shifts as explained above with reference to FIGS. 6A-6F. Thus, when in multiple changing magnetic fields that are rotating in phase, transducer 168 will produce the same voltage power output as if it were in a single changing magnetic field so long as the strength of the magnetic field(s) are sufficient to effect the voltage producing shifts of polarity within the transducer 168.

Graph 830 shows pulse energy of each pulse p plotted against rotational speed of the changing magnetic field, i.e., rotational speed of the rotor 164. As may be appreciated from graph 830, the pulse energy for the individual pulses p_{J}, p_{K} remains constant across all rotational speeds of the magnetic field, meaning that the voltage magnitude of each pulse p_{J}, p_{K} is unaffected by rotational speed of the magnetic field. However, as noted above, an increase in the rotational speed of the magnetic field decreases the time interval at which the pulses p_{J}, p_{K} are generated, so increased rotational speed of the rotor 164 produces more power as harvestable energy in the form of voltage pulses generated from the Wiegand inductor per unit of time increases. It is to be understood that graphs 810, 820, and 830 shown in FIG. 8 are intended to be illustrative only of general concepts and are not intended to convey actual values of the various properties plotted thereon.

FIG. 9 shows a graph of empirical data gathered from an actual non-limiting Wiegand inductor embodiment in response to a rotating magnetic field. Data was gathered using an oscilloscope. It is noted that voltage is shown directly as generated by the Wiegand inductor, prior to any voltage conditioning. That is, the voltage is shown as output by the transducer 168 prior to conditioning in the conversion box 170 (as shown in FIGS. 2A and 4A). Time, in units of milliseconds (each vertical bar represents 1.000 millisecond), is plotted on the x-axis and voltage, in units of volts (each horizontal bar represents 2.00 volts), is plotted on the y-axis. The graph shows three voltage pulses p1, p3, and a second cycle of p1 generated by the Wiegand inductor as shown in FIGS. 6A-6C. As noted herein in connection with FIGS. 6A-6F, the second and fourth voltage pulses p2, p4 happen to generate negligible energy is this case so that the second and fourth voltage pulses p2, p4 do not register on the oscilloscope at the above described voltage scale. The first cycle of pulse p1 has a maximum magnitude of approximately 6 volts. The next pulse p3 has a maximum magnitude of approximately (−6) volts, the value being negative as a result of the change in direction of the magnetic field illustrated in FIG. 6E. The second cycle of pulse p1 has a maximum magnitude of approximately 6 volts, the value changing back to positive as a result of the change in direction of the magnetic field as illustrated in FIG. 6B. As may be appreciated from FIG. 9, each voltage pulse p1, p3 includes an initial spike reaching the maximum magnitude followed by a series of oscillations gradually diminishing to zero voltage. FIG. 10 shows a close-up view of the first cycle of pulse p1 of FIG. 9 illustrating these oscillations (magnified such that each vertical bar represents 50.00 microseconds). The initial spike s1 reaches the maximum voltage of the first pulse p1 of approximately 6.0002 volts. Subsequent spikes s2-s11 oscillate about zero voltage and sequentially diminish in magnitude until the signal stabilizes at zero volts.

FIG. 11 provides a graphical illustration of the root mean square (RMS) voltage output of the transducer 169 in the form of a dynamo, which may be used as an alternative inductor embodiment to the Wiegand inductor as described herein. Graph 910 shows magnetic flux B plotted against time t. Two different curves L and M of magnetic flux, illustrating two different rates of change of an alternating magnetic field, are represented. Curve L has a relatively slower change in flux over time in comparison to curve M. Graph 920 shows root mean square voltage V_{L} associated with curve L and root mean square voltage Vu associated with curve M plotted against time t. Graph 920 illustrates root mean square voltage generation over time in relation to magnetic field changes when compared to graph 910. As may be appreciated from graph 920, the voltage V_{L}, Vu is output at a substantially constant root mean square value, in contrast to the series of voltage pulses p output by the Wiegand inductor (as shown in FIG. 8). The output voltage V_{L} associated with curve L is less than the output voltage V_{M} associated with curve M because the AC voltage output of the dynamo 169 and corresponding root mean square voltage output are dependent upon the rate of change of the magnetic field. This is in contrast to the pulsed output of the Wiegand inductor 168, in which the magnitude of the pulses p_{J}, p_{K} is not influenced by the rate of change of the magnetic field although the number of pulses generated per unit of time is effected by the rate of change of the magnetic field. (See FIG. 8) Graph 930 shows energy of the AC voltage plotted against rotational speed of the magnetic field, i.e., rotational speed of the rotor 164. As may be appreciated from graph 930, the mean electrical energy increases substantially linearly with rotational speed of the magnetic field because the amplitude and frequency of the AC voltage generated by the dynamo 169 will increase linearly in proportion to increase in rotational speed of the rotor 164. That is, increasing the rotational speed of the changing magnetic field increases the amplitude of root mean square voltage output of the dynamo 169 in a linear manner. This is in contrast to the Wiegand inductor of FIG. 8, in which the amplitude of voltage of each pulse p remains constant regardless of the rotational speed of the magnetic field, which means that the amplitude of pulses p1 remain constant, the amplitude of pulses p2 remain constant, the amplitude of pulses p3 remain constant and the amplitude of pulses p4 remain constant even though rotational speed of the changing magnetic field is increased. It is to be understood that graphs 910, 920, and 930 shown in FIG. 11 are intended to be illustrative only of general concepts and are not intended to convey actual values of the various properties plotted thereon.

When exposing a dynamo 169 to multiple changing magnetic fields at the same time, such as under conditions where the magnetic fields of curves L and M are superimposed on the dynamo 169, for example, the dynamo 169 will generate more voltage power in the form of a larger RMS voltage output. This occurs regardless of whether the magnetic fields of curves L and M are changing in phase or not.

FIG. 12 shows a graph of empirical data gathered from an actual non-limiting dynamo embodiment in response to a rotating magnetic field. Data was gathered using an oscilloscope. It is noted that voltage is shown directly as generated by the dynamo inductor, prior to any voltage conditioning. That is, the voltage is shown as output by the transducer 169 prior to conditioning in the conversion box 171 (as shown in FIGS. 2B and 4B). Time, in units of milliseconds (each vertical line representing 10 milliseconds), is plotted on the x-axis and voltage, in units of volts (each horizontal line representing 2.00 volts), is plotted on the y-axis. The graph shows that the output voltage follows a substantially uniform waveform w, in contrast to the periodic pulses p1, p3, p1 exhibited by the Wiegand inductor of FIGS. 8-10. In the example shown, the wave w has a magnitude of approximately 5.7 volts, and a frequency of approximately 51.962 Hz, and the root mean square voltage corresponding to this AC output is 1.839 volts. As noted in connection with FIG. 11, the magnitude of the RMS voltage is a function of the speed of the rotating magnetic field, so the wave w shown in FIG. 12 is representative only of a particular rotational speed of the magnetic field. Magnetic fields having a different rotational speed will cause the dynamo 169 to generate a root mean square voltage of a different magnitude. More specifically, magnetic fields having a rotational frequency of faster than about 52 Hz will generate a higher root mean square voltage and magnetic fields having a rotational frequency slower than about 52 Hz will generate a lower root mean square voltage.

While the foregoing description has been primarily directed to medical devices, in particular an extracorporeal circulation system 10, those skilled in the art will appreciate that the principles described herein may be equally applied to other technological fields. For example, the electrical circuits shown in FIGS. 2, 4, and 5, including the transducer 168 and associated components, may be implemented in a variety of fluid flow systems apart from extracorporeal circulation by removing the oxygenator 18 and replacing the blood pump 16 with a fluid pump suitable for the desired system that pumps a fluid other than blood. Likewise, the at least one sensor 180 could encompass any sensor or other low-power electronic component of the desired system. For example, the electrical circuits shown in FIGS. 2, 4, and 5, including the transducer 168 and associated components, may be implemented in a hemodialysis machine with the transducer 168 providing power for sensors associated with a dialyzer. Even more generally, the use of the transducer 168, in the form of a Wiegand inductor or dynamo, to harvest energy from an electric motor that induces a rotating or otherwise changing magnetic field, and using the harvested energy for powering sensors and other electrical components, will be understood by those skilled in the art to fall within the scope of the present disclosure.

While various examples of the present disclosure were provided in the foregoing description, those skilled in the art may make modifications and alterations to these examples without departing from the scope and spirit of the disclosure. For example, it is to be understood that features of various embodiments described herein may be adapted to other embodiments described herein. Accordingly, the foregoing description is intended to be illustrative rather than restrictive. The disclosure described hereinabove is defined by the appended claims, and all changes to the disclosure that fall within the meaning and the range of equivalency of the claims are to be embraced within their scope.

1-51. (canceled)

52. A fluid flow system, comprising:
a fluid pump comprising a pump rotor and an electric motor, the pump rotor being coupled to be rotated by the electric motor;
a transducer disposed in operative proximity to the pump rotor and configured to generate electrical energy in response to a first changing magnetic field associated with rotation of the pump rotor; and
at least one electric device is powered by electrical energy generated by the transducer.

53. The system of claim 52, wherein the electric motor comprises a motor rotor and a motor stator, the motor rotor configured to rotate relative to the motor stator in response to a second changing magnetic field associated with the motor stator and the motor rotor.

54. The system of claim 53, wherein the transducer is disposed in operative proximity to the motor stator and the motor rotor to generate electrical energy in response to the second changing magnetic field associated with the motor stator and the motor rotor.

55. The system of claim 53, wherein at least one of the motor rotor and the motor stator comprises permanent magnets,
wherein at least one of the motor rotor and the motor stator comprises electromagnets, and
wherein the electric motor is configured to periodically change a polarity of the electromagnets to cause rotation of the motor rotor.

56. The system of claim 52, wherein the transducer comprises a Wiegand inductor.

57. The system of claim 52, wherein the transducer comprises a dynamo.

58. The system of claim 52, wherein the at least one electric device comprises at least one of a fluid temperature sensor, a fluid pressure sensor, a flow rate sensor, and a distance sensor.

59. The system of claim 52, wherein the pump rotor is magnetically coupled to the electric motor so as to generate the first changing magnetic field when the pump rotor is rotated.

60. The system of claim 52, wherein a drive shaft couples the pump rotor to the electric motor so as to rotate the pump rotor to generate the first changing magnetic field when the pump rotor is rotated.

61. The system of claim 52, further comprising:
a controller programmed or configured to receive an output data signal from the at least one electric device.

62. The system of claim 61, wherein the at least one electric device is configured to wirelessly transmit the output data signal to the controller.

63. The system of claim 52, wherein the electrical energy generated by the transducer comprises a plurality of first voltage pulses, each first voltage pulse having a substantially constant voltage unaffected by changes to a rotational speed of the first changing magnetic field.

64. The system of claim 54, wherein the electrical energy generated by the transducer comprises a plurality of first voltage pulses, each first voltage pulse having a substantially constant voltage unaffected by changes to a rotational speed of the first and second changing magnetic fields.

65. The system of claim 63, wherein the electrical energy generated by the transducer comprises a plurality of second voltage pulses, each second voltage pulse having a substantially constant voltage unaffected by changes to the rotational speed of the first changing magnetic field, wherein the first voltage pulses are substantially different from the second voltage pulses.

66. The system of claim 52, further comprising:
a conversion box configured to store and condition the electrical energy generated by the transducer so that the electrical energy generated by the transducer is in a form suitable to power the at least one electric device.

67. The system of claim 52, wherein the fluid pump is a blood pump and the system is a medical device selected from the group consisting of a cardiopulmonary bypass machine, an extracorporeal membrane oxygenation machine, a pump assisted lung protection machine, and a hemodialysis machine.

68. A method for generating electrical energy to power at least one electronic device of a system, the method comprising the steps of:
in a fluid pump of the system comprising a magnetic field coupling a pump rotor and an electric motor, rotating the pump rotor to generate a first changing magnetic field associated with the pump rotor;
inducing a voltage in a transducer of the system as a result of rotating the first changing magnetic field, wherein the transducer comprises a Wiegand inductor and the induced voltage comprises a plurality of first voltage pulses, each first voltage pulse having a substantially constant voltage unaffected by changes to a rotational speed of the first changing magnetic field when rotating the first changing magnetic field; and
powering the at least one electronic device using the induced voltage as a source of electrical energy.

69. The method of claim 68, wherein the electric motor comprises a motor rotor and a motor stator, the motor rotor configured to rotate relative to the motor stator in response to a second changing magnetic field associated with the motor stator and the motor rotor, and the method further comprises the step of:
generating the second changing magnetic field so as to rotate the motor rotor relative to the motor stator.

70. The method of claim 69, further comprising:
inducing an additional voltage in the transducer as a result of rotating the second changing magnetic field.

71. The method of claim 69, wherein at least one of the motor rotor and the motor stator comprises permanent magnets,
wherein at least one of the motor rotor and the motor stator comprises electromagnets, and
wherein the electric motor is configured to periodically change a polarity of the electromagnets to cause rotation of the motor rotor.

72. The method of claim 68, wherein the induced voltage comprises a plurality of second voltage pulses, each second voltage pulse having a substantially constant voltage unaffected by changes to the rotational speed of the first changing magnetic field, wherein the first voltage pulses are substantially different from the second voltage pulses.

73. The method of claim 68, further comprising the step of:
conditioning the electrical energy generated by the transducer, wherein the electrical energy is conditioned with a conversion box so that the induced voltage generated by the transducer is in a form suitable to power the at least one electronic device.

74. The method of claim 68, wherein the fluid pump is a blood pump and the system is a medical device selected from the group consisting of a cardiopulmonary bypass machine, an extracorporeal membrane oxygenation machine, a pump assisted lung protection machine, and a hemodialysis machine, and the at least one electronic device powered by the electrical energy is selected from the group consisting of a blood temperature sensor, a blood flow sensor, a blood pressure sensor, and a distance sensor.

75. An electric system, comprising:
an electric motor;
a rotor coupled to be rotated by the electric motor;
a transducer disposed in operative proximity to the rotor and configured to generate electrical energy in response to a first changing magnetic field associated with rotation of the rotor; and
at least one electronic device is powered by electrical energy generated by the transducer.

76. The system of claim 75, wherein the electric motor comprises a motor rotor and a motor stator, the motor rotor configured to rotate relative to the motor stator in response to a second changing magnetic field associated with the motor stator and the motor rotor.

77. The system of claim 76, wherein the transducer is disposed in operative proximity to the motor stator and the motor rotor to generate electrical energy in response to the second changing magnetic field associated with the motor stator and the motor rotor.

78. The system of claim 76, wherein at least one of the motor rotor and the motor stator comprises permanent magnets,
wherein at least one of the motor rotor and the motor stator comprises electromagnets, and
wherein the electric motor is configured to periodically change a polarity of the electromagnets to cause rotation of the motor rotor.

79. The system of claim 75, wherein the transducer comprises a Wiegand inductor.

80. The system of claim 75, wherein the electrical energy generated by the transducer comprises a plurality of first voltage pulses, each first voltage pulse having a substantially constant voltage unaffected by changes to a rotational speed of the first changing magnetic field.

81. The system of claim 77, wherein the electrical energy generated by the transducer comprises a plurality of first voltage pulses, each first voltage pulse having a substantially constant voltage unaffected by changes to a rotational speed of the first and second changing magnetic fields.

82. The system of claim 80, wherein the electrical energy generated by the transducer comprises a plurality of second voltage pulses, each second voltage pulse having a substantially constant voltage unaffected by changes to the rotational speed of the first changing magnetic field, wherein the first voltage pulses are substantially different from the second voltage pulses.

83. The system of claim 75, further comprising:
a conversion box configured to store and condition the electrical energy generated by the transducer so that the electrical energy generated by the transducer is in a form suitable to power the at least one electronic device.

84. The system of claim 75, wherein the electric motor is a component of a blood pump and the system is a medical device selected from the group consisting of a cardiopulmonary bypass machine, an extracorporeal membrane oxygenation machine, a pump assisted lung protection machine, and a hemodialysis machine, and the at least one electronic device powered by the electrical energy is selected from the group consisting of a blood temperature sensor, a blood flow sensor, a blood pressure sensor, and a distance sensor.
